(19) United States
(12) Patent Application Publication
(10) Pub. No.: US 2023/0357135 A1
(43) Pub. Date: Nov. 9, 2023

(54) CRYSTALLINE FORMS OF OMADACYCLINE, METHODS OF SYNTHESIS THEREOF AND METHODS OF USE THEREOF

(71) Applicant: Paratek Pharmaceuticals, Inc., Boston, MA (US)

(72) Inventors: Tadeusz Warchol, Northborough, MA (US); Sean M. Johnston, Doylestown, PA (US)

(21) Appl. No.: 18/009,670

(22) PCT Filed: Jun. 11, 2021
(86) PCT No.: PCT/US2021/036977

§ 371 Date: Dec. 9, 2022

Related U.S. Application Data
(60) Provisional application No. 63/037,807, filed on Jun. 11, 2020.

(51) Int. Cl. C07C 237/26

(52) U.S. Cl. CPC C07C 237/26; C07B 2200/13; C07C 2603/46

(57) ABSTRACT

The present invention provides omadacycline crystalline freebase and methods of synthesis thereof. The present invention also provides pharmaceutical compositions comprising omadacycline crystalline freebase and methods of use thereof for treating bacterial infections. The present invention also provides methods of purifying crude omadacycline freebase that comprise crystallization to produce crystalline omadacycline freebase. The omadacycline crystalline freebase may also be used to prepare salts of omadacycline, e.g., a tosylate salt.

### RELATED APPLICATIONS

This application claims priority to U.S. Provisional Application No. 63/037,807, filed on Jun. 11, 2020, the entire contents of which are hereby incorporated herein by reference.

### INTRODUCTION

Omadacycline (also known as OMC or PTK 0796) is a 9-aminomethyl tetracycline derivative that is currently in advanced clinical development for the treatment of various bacterial infections. Structure of omadacycline is shown below:

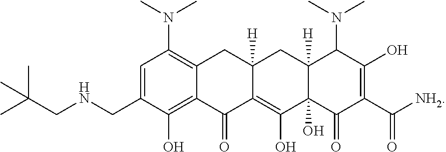 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

Chemical synthesis of omadacycline has been previously described, e.g., in U.S. Pat. Nos. 9,434,680, 9,522,872 and 8,383,610, the entire contents of each of which are incorporated herein by reference. An exemplary procedure for the synthesis of omadacycline is shown in Scheme 1 below. In this procedure, minocycline (A) is used as the starting material, which in the first step is subjected to alkylation at the 9-position with N-(hydroxymethyl)phtalimide. Product of this conversion (B) is reacted with methylamine, which results in deprotection of amine groups and affords intermediate (C). In the next step, intermediate (C) is reacted with trimethylacetaldehyde under reductive alkylation conditions to yield crude omadacycline in the freebase form.

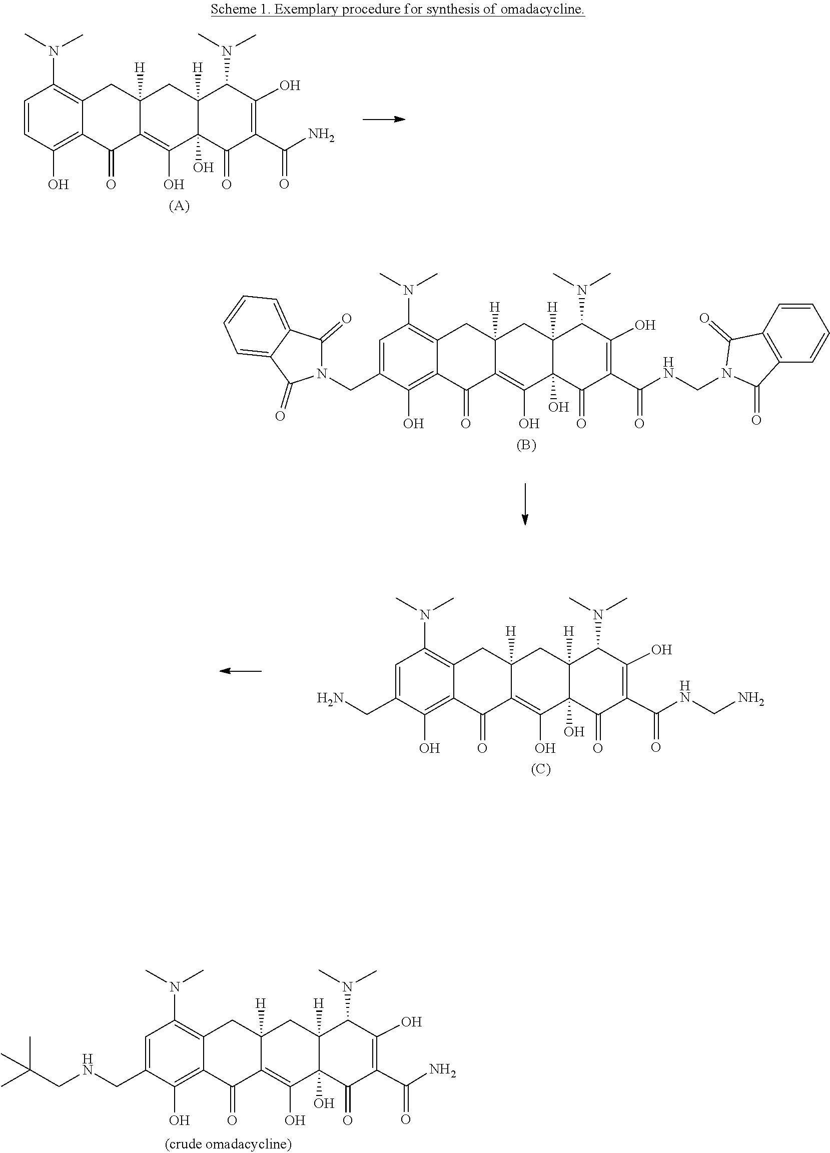

Subsequent to synthesis, crude omadacycline freebase is purified and converted to a tosylate salt form which, in turn, is used for preparing pharmaceutical compositions of omadacycline. Purification of crude omadacycline freebase involves using high performance liquid chromatography (HPLC) and collecting HPLC fractions containing purified omadacycline freebase. Subsequently, omadacycline freebase in the HPLC fractions are concentrated by extraction with dichloromethane. This purification procedure may be time consuming, e.g., requiring more than 70 hours of processing time in order to generate one kilogram of pure omadacycline. It exposes omadacycline to prolonged periods of higher temperatures during solvent evaporation, which leads to degradation. Furthermore, this purification procedure requires large amounts of solvent, thereby generating substantial waste. Therefore, easier and more efficient procedures for purifying omadacycline freebase to yield a high purity product are desired. Additionally, novel pure forms of omadacycline that may be formulated into pharmaceutical compositions are also needed.

### SUMMARY OF THE INVENTION

Accordingly, in some aspects, the present invention provides methods for purifying omadacycline, e.g., crude omadacycline freebase. The methods include, in some embodiments, a modified HPLC procedure that utilizes a modifier, e.g., acetic acid, and results in a faster and more efficient purification of crude omadacycline freebase than the previously used methods.

The methods for purifying omadacycline freebase of the present invention also include, in some embodiments, concentrating HPLC fractions containing omadacycline freebase using nanofiltration prior to omadacycline crystallization. The use of nanofiltration replaces extraction of HPLC fractions with a solvent, e.g., dichloromethane, thereby eliminating the requirement for using large amounts of a toxic solvent in the purification process. The use of nanofiltration also avoids exposure of omadacycline to higher temperatures during prolonged periods, which may lead to degradation of omadacycline. Optionally, if extraction with a solvent, e.g., dichloromethane, is used after nanofiltration, nanofiltration leads to a significant reduction of the amount of solvent required for extraction of omadacycline as compared to extraction performed without nanofiltration. The methods of purifying omadacycline described herein also include, in some embodiments, crystallizing omadacycline freebase, e.g., after HPLC purification, nanofiltration and optional extraction, to yield a highly pure omadacycline crystalline freebase, with significantly lower amounts of impurities than in omadacycline generated using the previous methods.

The novel purification methods of the present invention resulted, for the first time, in synthesis of omadacycline crystalline freebase. Thus, in some embodiments, the present invention also provides omadacycline crystalline freebase, pharmaceutical compositions comprising omadacycline crystalline freebase and methods of treating bacterial infections using omadacycline crystalline freebase.

The omadacycline crystalline freebase of the present invention may also be used to synthesize highly pure omadacycline tosylate salt. Accordingly, in some embodiments, the present invention also provides methods of preparing a tosylate salt of omadacycline from omadacycline crystalline freebase. The present invention also provides omadacycline tosylate salt prepared by the methods of the present invention.

Accordingly, in some aspects, the present invention provides a crystalline form of freebase of omadacycline, wherein omadacycline is represented by formula (1):

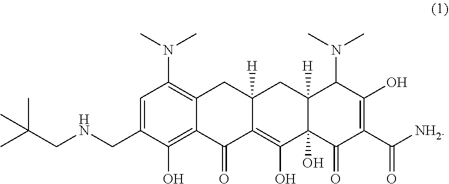 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

In a further embodiment, omadacycline is represented by formula (2):

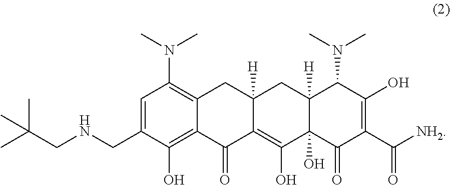 [SMILES: CN(C)c1cc(CNCC(C)(C)C)c(O)c2c1C[C@H]1C[C@H]3[C@H](N(C)C)C(O)=C(C(N)=O)C(=O)[C@@]3(O)C(O)=C1C2=O]

In some aspects, the present invention also provides a polymorph of the crystalline form of freebase of omadacycline characterized by an X-ray powder diffraction pattern that includes at least one peak selected from the group consisting of:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

In some embodiments, the polymorph is characterized by an X-ray powder diffraction pattern that includes the following peaks:
a peak at approximately 7.25° 2θ;
a peak at approximately 7.37° 2θ;
a peak at approximately 12.58° 2θ;
a peak at approximately 12.81° 2θ;
a peak at approximately 16.44° 2θ; and
a peak at approximately 17.86° 2θ.

In some embodiments, the polymorph is characterized by an X-ray powder diffraction pattern that includes the following peaks:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

In some aspects, the present invention also provides a method of preparing the polymorph as described above, comprising crystallizing freebase form of omadacycline from a solvent system that comprises an organic solvent and water. In some embodiments, the organic solvent and water are present in the solvent system at a ratio ranging from about 5:95 v/v to about 95:5 v/v organic solvent:water. In further embodiments, the organic solvent is selected from the group consisting of acetonitrile, acetone, isopropyl alcohol and methyl ethyl ketone. In one specific embodiment, the organic solvent is acetone.

In some aspects, the present invention also provides a polymorph of crystalline form of freebase of omadacycline prepared by the method as described above. For example, in some embodiments, the present invention provides a polymorph of crystalline form of freebase of omadacycline prepared by a method comprising: crystallizing freebase form of omadacycline from a solvent system that comprises an organic solvent and water.

In some embodiments, the organic solvent and water are present in the solvent system at a ratio ranging from about 5:95 v/v to about 95:5 v/v organic solvent:water. In further embodiments, the organic solvent is selected from the group consisting of acetonitrile, acetone, isopropyl alcohol and methyl ethyl ketone. In one specific embodiment, the organic solvent is acetone.

In some embodiments, the polymorph is characterized by an X-ray powder diffraction pattern that includes at least one peak selected from the group consisting of:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

In some embodiments, the polymorph is characterized by an X-ray powder diffraction pattern that includes the following peaks:
a peak at approximately 7.25° 2θ;
a peak at approximately 7.37° 2θ;
a peak at approximately 12.58° 2θ;
a peak at approximately 12.81° 2θ;
a peak at approximately 16.44° 2θ; and
a peak at approximately 17.86° 2θ.

In some embodiments, the polymorph is characterized by an X-ray powder diffraction pattern that includes the following peaks:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

In some embodiments, the acetone and water are present in the solvent system at a ratio of about 50:50 v/v acetone:water. In certain aspects, the organic solvent is selected from the group consisting of isopropanol, acetonitrile and methyl ethyl ketone.

In some aspects, the present invention also provides a method of purifying freebase form of omadacycline, wherein the omadacycline is represented by formula (1):

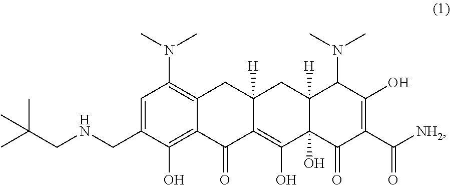 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

the method comprising
subjecting a solution comprising crude freebase form of omadacycline to purification by high performance liquid chromatography (HPLC), wherein the HPLC comprises the use of a modifier selected from the group consisting of a strong acid which is not methyl sulfonic acid (e.g., hydrochloric acid), a weak acid and an organic amine, thereby obtaining a solution comprising HPLC-purified freebase form of omadacycline.

In some embodiments, the modifier is a weak acid, and wherein the weak acid is selected from the group consisting of oxalic acid, methanesulfonic acid, trifluoracetic acid, sulfurous acid, phosphoric acid, nitrous acid, hydrofluoric acid, benzoic acid, acetic acid and formic acid. In some embodiments, the weak acid is selected from the group consisting of oxalic acid, methanesulfonic acid, trifluoracetic acid, benzoic acid, acetic acid and formic acid. In a specific embodiment, the weak acid is acetic acid. In some embodiments, the modifier is added to the mobile phase during HPLC; or wherein the modifier is added to the solution comprising crude freebase form of omadacycline prior to loading onto HPLC column. In some embodiments, the mobile phase comprises elution buffer A and elution buffer B; wherein the elution buffer A comprises water and acetonitrile and/or wherein the elution buffer B comprises acetonitrile.

In some aspects, the amount of beta epimer impurity of omadacycline in the solution comprising HPLC-purified freebase form of omadacycline is at least 5 times lower than the amount of the beta epimer impurity of omadacycline present in the solution comprising crude freebase form of omadacycline.

In some embodiments, the methods of the present invention further comprise concentrating the solution comprising the HPLC-purified free base form of omadacycline using nanofiltration, wherein the nanofiltration comprises filtering the solution comprising HPLC-purified free base form of omadacycline through a membrane to form a filtrate and a retentate, wherein the retentate is a concentrated solution comprising HPLC-purified free base form of omadacycline. In one embodiment, the methods further comprise collecting the retentate.

In some embodiments, the methods of the present invention further comprise adding an antioxidant to the solution comprising HPLC-purified free base form of omadacycline prior to nanofiltration. In further embodiments, the antioxidant is added in an amount sufficient to achieve a concentration of the antioxidant in the solution of about 0.01% to about 0.5% w/v.

In some embodiments, the membrane has a molecular weight cut-off (MWCO) ranging from about 150 to about 500 Daltons. In some embodiments, the concentration of omadacycline in the retentate is at least about 2 times greater, e.g., at least 3 times, at least 4 times, at least 5 times, at least 6 times, at least 8 times or at least 10 times greater, than the concentration of omadacycline in the solution comprising HPLC-purified free base form of omadacycline.

In some embodiments, the methods of the present invention further comprise crystallizing freebase form of omadacycline, thereby obtaining a crystalline form of the freebase of omadacycline. In some embodiments, the freebase form of omadacycline is crystalized from a solvent system that comprises an organic solvent and water. In further embodiments, the organic solvent and water are present in the solvent system at a ratio ranging from about 5:95 v/v to about 95:5 v/v organic solvent:water. In some embodiments, the organic solvent is selected from the group consisting of a nitrile, an alcohol, a ketone and an ether. For example, the organic solvent may be selected from the group consisting of acetonitrile, acetone, isopropyl alcohol and methyl ethyl ketone. In a specific embodiment, the organic solvent is acetone.

In some embodiments, the acetone and water are present in the solvent system at a ratio of about 50:50 v/v acetone:water. In some embodiments, the organic solvent is selected from the group consisting of isopropanol, acetonitrile and methyl ethyl ketone.

In some embodiments, the crystalline form of the freebase of omadacycline obtained by the methods of the present invention is a polymorph characterized by an X-ray powder diffraction pattern that includes at least one peak selected from the group consisting of:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

In some aspects, the present invention also provides a method of preparing a tosylate salt of omadacycline, wherein the omadacycline is represented by formula (1):

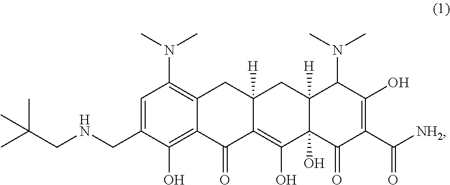 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

the method comprising:
purifying a freebase form of omadacycline by the methods as described above, thereby obtaining a purified freebase form of omadacycline; and
reacting the purified freebase form of omadacycline in a tosylation reaction, thereby obtaining a tosylate salt of omadacycline.

In some embodiments, the present invention also provides a method of preparing a tosylate salt of omadacycline, wherein the omadacycline is represented by formula (1):

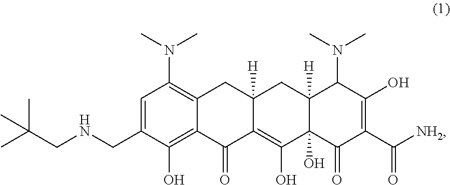 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

the method comprising: crystallizing freebase form of omadacycline, thereby obtaining a crystalline form of the freebase of omadacycline; and reacting the crystalline form of the freebase of omadacycline in a tosylation reaction, thereby obtaining a tosylate salt of omadacycline.

In some embodiments, the freebase form of omadacycline is crystalized from a solvent system that comprises an organic solvent and water. In some embodiments, the organic solvent and water are present in the solvent system at a ratio ranging from about 5:95 v/v to about 95:5 v/v organic solvent:water. In some embodiments, the organic solvent is selected from the group consisting of acetonitrile, acetone, isopropyl alcohol, methyl ethyl ketone, t-butyl methyl ether, ethyl acetate, toluene and tetrahydrofuran. In one specific embodiment, the organic solvent is acetone.

In some embodiments, the acetone and water are present in the solvent system at a ratio of about 50:50 v/v acetone:water. In some embodiments, the organic solvent is selected from the group consisting of isopropanol, acetonitrile and methyl ethyl ketone.

In some embodiments, the crystalline form of the freebase of omadacycline is a polymorph characterized by an X-ray powder diffraction pattern that includes at least one peak selected from the group consisting of:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

In some embodiments, the methods of the invention comprise subjecting a solution comprising crude freebase form of omadacycline to purification by high performance liquid chromatography (HPLC), wherein the HPLC comprises the use of a modifier selected from the group consisting of a strong acid which is not methyl sulfonic acid (e.g., hydrochloric acid), a weak acid and an organic amine, thereby obtaining a solution comprising HPLC-purified freebase form of omadacycline; and crystallizing freebase form of omadacycline from the solution comprising HPLC-purified freebase from of omadacycline, thereby obtaining a crystalline form of the freebase of omadacycline; and reacting the crystalline form of the freebase of omadacycline in a tosylation reaction, thereby obtaining a tosylate salt of omadacycline.

In some embodiments, the modifier is a weak acid, wherein the weak acid is selected from the group consisting of oxalic acid, methanesulfonic acid, trifluoracetic acid, sulfurous acid, phosphoric acid, nitrous acid, hydrofluoric acid, benzoic acid, acetic acid and formic acid. In some embodiments, the weak acid is selected from the group consisting of oxalic acid, methanesulfonic acid, trifluoracetic acid, benzoic acid, acetic acid and formic acid. In one specific embodiment, the weak acid is acetic acid.

In some embodiments, the modifier is added to the mobile phase during HPLC; or the modifier is added to the solution comprising crude freebase form of omadacycline prior to loading onto HPLC column. In some embodiments, the mobile phase comprises elution buffer A and elution buffer B; wherein the elution buffer A comprises water and acetonitrile and/or wherein the elution buffer B comprises acetonitrile.

In some embodiments, the methods of the present invention further comprise concentrating the solution comprising the HPLC-purified free base form of omadacycline using nanofiltration, wherein the nanofiltration comprises filtering the solution comprising HPLC-purified free base form of omadacycline through a membrane to form a filtrate and a retentate, wherein the retentate is a concentrated solution comprising HPLC-purified free base form of omadacycline. In some aspects, the methods of the invention further comprise collecting the retentate.

In some embodiments, the methods of the invention further comprise adding an antioxidant to the solution comprising HPLC-purified free base form of omadacycline prior to nanofiltration. In further embodiments, the antioxidant is added in an amount sufficient to achieve a concentration of the antioxidant in the solution of about 0.01% to about 0.5% w/v. In some embodiments, the membrane used in nanofiltration has a molecular weight cut-off (MWCO) ranging from about 150 to about 500 Daltons.

In some aspects, the present invention also provides methods of preparing a tosylate salt of omadacycline, wherein the omadacycline is represented by formula (1):

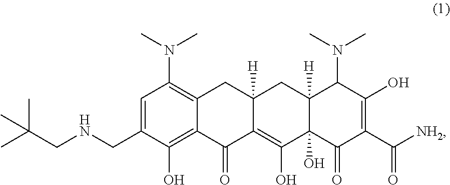 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

the method comprising:
subjecting a solution comprising crude freebase form of omadacycline to purification by high performance liquid chromatography (HPLC), wherein the HPLC comprises the use of a modifier selected from the group consisting of a weak acid and an organic amine, thereby obtaining a solution comprising HPLC-purified freebase form of omadacycline;
concentrating the solution comprising the HPLC-purified freebase form of omadacycline using nanofiltration, wherein the nanofiltration comprises filtering the solution comprising HPLC-purified free base form of omadacycline through a membrane to form a filtrate and a retentate, wherein the retentate is a concentrated solution comprising HPLC-purified free base form of omadacycline;
crystallizing freebase form of omadacycline from the concentrated solution comprising HPLC-purified freebase from of omadacycline, thereby obtaining a crystalline form of the freebase of omadacycline; and
reacting the crystalline form of the freebase of omadacycline in a tosylation reaction, thereby obtaining a tosylate salt of omadacycline.

In some aspects, the present invention also provides a tosylate salt of omadacycline, wherein the omadacycline is represented by formula (1):

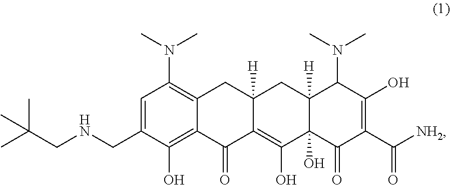 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

obtained by the methods as described above.

In some aspects, the present invention also provides a crystalline tosylate salt of omadacycline, wherein the omadacycline is represented by formula (1):

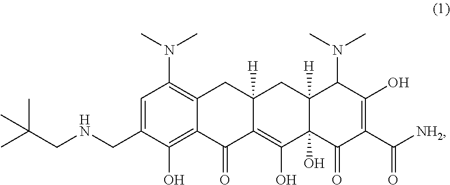 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

obtained by the methods as described above. In a further embodiment, the present invention also provides a polymorph of the crystalline tosylate salt, e.g., Form 1 polymorph or a Form 3 polymorph.

In some aspects, the present invention also provides a pharmaceutical composition comprising the crystalline form of freebase of omadacycline as described above and a pharmaceutically acceptable carrier. In some embodiments, the present invention provides a pharmaceutical composition comprising the polymorph of the crystalline form of freebase of omadacycline as described above and a pharmaceutically acceptable carrier.

In some aspects, the present invention also provides a pharmaceutical composition comprising the tosylate salt of omadacycline as described above and a pharmaceutically acceptable carrier. In some embodiments, the present invention also provides a pharmaceutical composition comprising the crystalline tosylate salt of omadacycline as described above and a pharmaceutically acceptable carrier. In some embodiments, the present invention also provides a pharmaceutical composition comprising the polymorph of the crystalline tosylate salt of omadacycline as describe above and a pharmaceutically acceptable carrier.

In some embodiments, the pharmaceutical composition is in a tablet form. In other embodiments, the pharmaceutical composition is an injectable formulation in the form of a lyophilized powder.

In some aspects, the present invention also provides a method of treating or preventing a bacterial infection in a subject in need thereof that comprises administering to the subject an effective amount of the crystalline form of freebase of omadacycline, or the pharmaceutical composition as described above.

In some aspects, the present invention also provides a method of treating or preventing a bacterial infection in a subject in need thereof that comprises administering to the subject an effective amount of the polymorph of the crystalline form of freebase of omadacycline as, or the pharmaceutical composition as described above.

In some aspects, the present invention also provides a method of treating or preventing a bacterial infection in a subject in need thereof that comprises administering to the subject an effective amount of the tosylate salt of omadacycline, or the pharmaceutical composition as described above.

In some aspects, the present invention also provides a method of treating or preventing a bacterial infection in a subject in need thereof that comprises administering to the subject an effective amount of the crystalline tosylate salt of omadacycline, or the pharmaceutical composition as described above.

In some aspects, the present invention also provides a method of treating or preventing a bacterial infection in a subject in need thereof that comprises administering to the subject an effective amount of the polymorph of the crystalline tosylate salt of omadacycline, or the pharmaceutical composition as described above.

In some embodiments, the bacterial infection is caused by a Gram-positive or a Gram-negative bacteria. In some embodiments, the bacterial infection is caused by a bacteria that is resistant to other tetracycline compounds. In some aspects, the bacterial infection is caused by a bacteria of a species selected from the group consisting of K. pneumoniae, Salmonella, E. hirae, A. baumanii, B. catarrhalis, H. influenza, P. aeruginosa, E. faecium, E. coli, S. aureus and E. faecalis.

In some embodiments, the bacterial infection is an acute bacterial skin structure infection (ABSSSI). In further embodiments, the ABSSSI is caused by a bacteria of a species selected from the group consisting of Staphylococcus aureus (methicillin-susceptible and -resistant isolates), including cases with concurrent bacteremia, Staphylococcus lugdunensis, Streptococcus pyogenes, Streptococcus agalactiae, Streptococcus anginosus grp. (includes S. anginosus, S. intermedius, and S. constellatus), Streptococcus mitis, Enterococcus faecalis (vancomycin-susceptible isolates), Enterobacter cloacae, Klebsiella pneumoniae, Prevotella melaninogenica, and Finegoldia magna.

In some embodiments, the bacterial infection is a community-acquired bacterial pneumonia (CABP). In further embodiments, the CABP is caused by a bacteria of a species selected from the group consisting of Streptococcus pneumoniae (penicillin-susceptible and -resistant isolates, macrolide-resistant isolates), including cases with concurrent bacteremia, Staphylococcus aureus (methicillin-susceptible isolates), Haemophilus influenzae (beta-lactamase negative and positive isolates), Haemophilus parainfluenzae, Klebsiella pneumoniae, Legionella pneumophila, Mycoplasma pneumoniae, and Chlamydophila pneumoniae.

In some embodiments, the bacterial infection is caused by a bacterial of a species C. difficile. In some aspects, the bacterial infection is caused by a mycobacteria.

### BRIEF DESCRIPTION OF THE FIGURES

FIG. 1 is a typical XRPD spectrum of omadacycline crystalline freebase.

FIG. 2 is an XRPD spectrum of omadacycline freebase crystallized from a solvent system comprising acetonitrile and water (wet acetonitrile).

FIG. 3 is an XRPD spectrum of omadacycline freebase crystallized from a solvent system comprising isopropanol and water (wet isopropanol).

FIG. 4 is an XRPD spectrum of omadacycline freebase crystallized from a solvent system comprising 2-butanone and water (wet 2-butanone).

FIG. 5 is an XRPD spectrum of control omadacycline crystalline freebase.

FIG. 6 is a schematic illustration of a process for preparing omadacycline crystalline freebase.

FIG. 7 is a schematic illustration of a process for preparing crystalline tosylate salt of omadacycline that comprises preparing omadacycline crystalline freebase.

### DETAILED DESCRIPTION OF THE INVENTION

### Crystalline Free Base of Omadacycline

The present invention provides a crystalline form of omadacycline freebase. Omadacycline is a 9-aminomethyl tetracycline derivative being developed as a first-line agent for empiric therapy primarily for serious community acquired infections, such as acute bacterial skin and skin structure infections (ABSSSIs), moderate to severe community-acquired bacterial pneumonia (CABP) and complicated urinary tract infections (cUTI). The name “omadacycline” may be used herein interchangeably with the name “OMC”, “PTK 0796” or “Compound 1”. In some examples, omadacycline may be represented by formula (1):

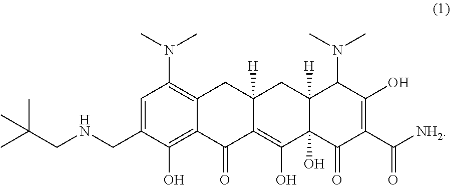 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

In some examples omadacycline may be represented by formula (2):

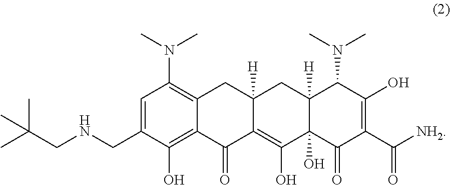 [SMILES: CN(C)c1cc(CNCC(C)(C)C)c(O)c2c1C[C@H]1C[C@H]3[C@H](N(C)C)C(O)=C(C(N)=O)C(=O)[C@@]3(O)C(O)=C1C2=O]

In its non-crystalline form, omadacycline is a yellow amorphous solid that may be particularly unstable upon exposure to air, light and/or moisture. Thus, in its solid form, omadacycline must be stored at temperatures below 0° C. and with limited exposure to air, light and moisture. Outside of these limited exposure conditions, omadacycline may degrade to produce degradation products, e.g., air degradation products represented by formula (3) and formula (4), as well as the 4-epi-isomer represented by formula (5).

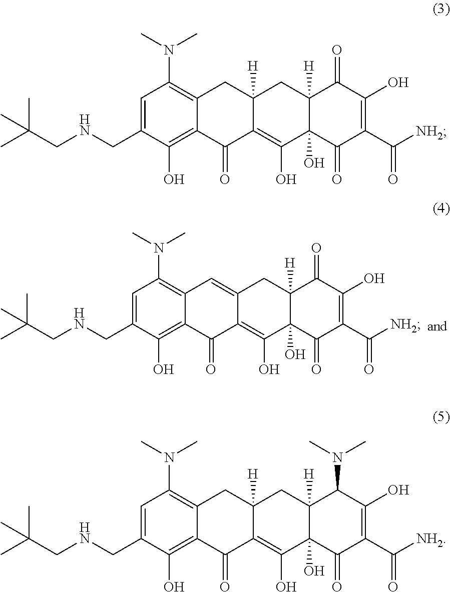 [SMILES: CN(C)c1cc(CNCC(C)(C)C)c(O)c2c1C[C@H]1C[C@H]3C(=O)C(O)=C(C(N)=O)C(=O)[C@@]3(O)C(O)=C1C2=O ; CN(C)c1cc(CNCC(C)(C)C)c(O)c2c1C=C1C[C@H]3C(=O)C(O)=C(C(N)=O)C(=O)[C@@]3(O)C(O)=C1C2=O ; CN(C)c1cc(CNCC(C)(C)C)c(O)c2c1C[C@H]1C[C@H]3[C@@H](N(C)C)C(O)=C(C(N)=O)C(=O)[C@@]3(O)C(O)=C1C2=O]

The freebase and certain pharmaceutically acceptable salts of omadacycline are described in U.S. Pat. No. 7,553,828, and certain crystalline salts of omadacycline are described in U.S. Pat. No. 8,383,610, the entire contents of each of which are incorporated herein by reference. However, prior to this disclosure, no crystalline forms, or polymorphs of crystalline forms, of omadacycline freebase were known.

Therefore, the present invention provides crystalline forms of freebase of omadacycline (Compound 1). The terms “crystalline” or “crystalline form”, as used herein, refer to a solid form of omadacycline in which atoms are arranged in regular, repeating patterns. In some embodiments, the term “crystalline” encompasses a polymorphic form or a non-amorphous form of omadacycline, without distinction.

The terms “amorphous” or “amorphous form”, as used herein, refer to a non-crystalline form of a substance, e.g., solid forms without a regular atomic arrangement.

The terms “polymorph” or “polymorphic form”, as used herein, refer to an organized structure involving only molecules of the solute and having a characteristic crystalline signature. These terms may refer to different crystalline forms of the same molecule. Different polymorphs may have different physical properties such as, for example, melting temperature, heat of fusion, solubility, dissolution rate and/or vibrational spectra as a result of different arrangements or conformations of the molecules in the crystal lattice. The differences in physical properties exhibited by different polymorphs may affect parameters important for pharmaceutical substances, such as storage stability, compressibility and density (important in formulation and product manufacturing), and dissolution rate (an important factor in bioavailability).

Differences in stabilities of different polymorphs may also result from differences in chemical reactivity (e.g., different susceptibility to oxidation). Thus, a dosage form comprised of one polymorph may discolor more rapidly than a dosage form comprised of a different polymorph of the same substance. Differences in stabilities of different polymorphs may also result from differences in mechanical properties (e.g., tablets may crumble on storage as a kinetically favored polymorph converts to a thermodynamically more stable polymorph); or from differences in both chemical and mechanical properties (e.g., tablets of one polymorph are more susceptible to breakdown at high humidity). As a result of solubility/dissolution differences, in extreme cases, some polymorphic transitions may result in loss of potency or, at the other extreme, toxicity. In addition, physical properties of the crystal may be important in processing. For example, different polymorphs of the same substances may exhibit differences in their propensity to form solvates or in their particle shape and size distributions, affecting purification (e.g., one polymorph may be difficult to filter and wash free of impurities than another polymorph).

Polymorphs of a molecule may be obtained by a number of methods, as known in the art. Such methods may include, but are not limited to, melt recrystallization, melt cooling, solvent recrystallization, desolvation, rapid evaporation, rapid cooling, slow cooling, vapor diffusion and sublimation. Techniques for characterizing polymorphs may include, but are not limited to, differential scanning calorimetry (DSC), X-ray powder diffractometry (XRPD), single crystal X-ray diffractometry, vibrational spectroscopy, e.g., IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies. Specifically, XRPD is a technique used to characterize the crystallographic structure, size, and preferred orientation in polycrystalline or powdered solid samples. This diffraction is also used to characterize heterogeneous solid mixtures to determine the percent of crystalline compounds present and can provide structural information on unknown materials. The term “X-ray powder diffraction pattern, used herein interchangeably with the term “XRPD pattern” refers to a graphical representation of the data collected by XRPD analysis.

In some embodiments, the present invention provides a polymorph of crystalline form of a freebase of omadacycline, also referred to herein as a “polymorph of omadacycline crystalline freebase”. In some embodiments, the polymorph of omadacycline crystalline freebase is characterized by an X-ray powder diffraction pattern (XRPD pattern) that includes at least one peak selected from the group consisting of:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

The term “peak”, as used herein, refers to a peak in the XRPD pattern having an intensity at least 20%, e.g., at least 30%, at least 40%, at least 50% or at least 100% greater than the baseline noise.

The terms “approximately” or “about”, as used herein in reference to a peak in an XRPD pattern, refer to the XRPD pattern in which the peak appears within 0.5° 2θ, e.g., within 0.4, 0.3, 0.2, 0.1, 0.05 or 0.01° 20 of a given ° 20 value.

In some embodiments, the present invention provides a polymorph of omadacycline crystalline freebase that is characterized by an X-ray powder diffraction pattern (XRPD pattern) that includes at least two peaks selected from the group of peaks as listed above.

In some embodiments, the present invention provides a polymorph of the crystalline form of omadacycline that is characterized by an X-ray powder diffraction pattern (XRPD pattern) that includes at least three peaks selected from the group of peaks as listed above.

In some embodiments, the present invention provides a polymorph of omadacycline crystalline freebase that is characterized by an X-ray powder diffraction pattern (XRPD pattern) that includes at least four peaks selected from the group of peaks as listed above.

In some embodiments, the present invention provides a polymorph of omadacycline crystalline freebase that is characterized by an X-ray powder diffraction pattern (XRPD pattern) that includes at least five peaks selected from the group of peaks as listed above.

In some embodiments, the present invention provides a polymorph of omadacycline crystalline freebase that is characterized by an X-ray powder diffraction pattern (XRPD pattern) that includes at least six peaks selected from the group of peaks as listed above.

In some embodiments, the present invention provides a polymorph of omadacycline crystalline freebase that is characterized by an X-ray powder diffraction pattern (XRPD pattern) that includes at least seven peaks selected from the group of peaks as listed above.

In some embodiments, the present invention provides a polymorph of omadacycline crystalline freebase that is characterized by an X-ray powder diffraction pattern (XRPD pattern) that includes at least eight peaks selected from the group of peaks as listed above.

In some embodiments, the present invention provides a polymorph of omadacycline crystalline freebase that is characterized by an X-ray powder diffraction pattern (XRPD pattern) that includes at least nine peaks selected from the group of peaks as listed above.

In some embodiments, the present invention provides a polymorph of omadacycline crystalline freebase that is characterized by an X-ray powder diffraction pattern (XRPD pattern) that includes at least ten peaks selected from the group of peaks as listed above.

In some embodiments, the present invention provides a polymorph of omadacycline crystalline freebase that is characterized by an X-ray powder diffraction pattern (XRPD pattern) that includes at least eleven peaks selected from the group of peaks as listed above.

In some embodiments, the present invention provides a polymorph of omadacycline crystalline freebase that is characterized by an X-ray powder diffraction pattern (XRPD pattern) that includes all peaks as listed above.

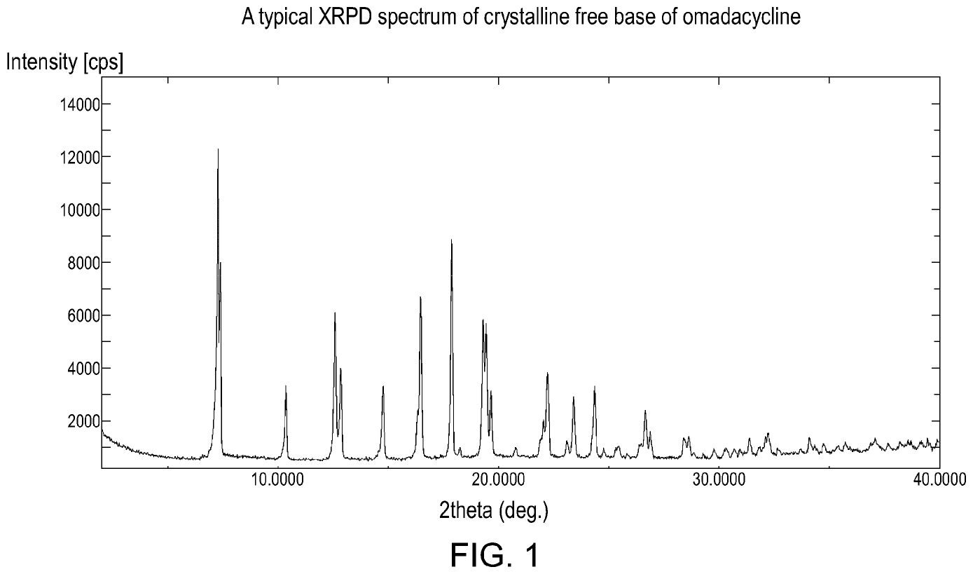

In some embodiments, the present invention provides a polymorph of omadacycline crystalline freebase that is characterized by an X-ray powder diffraction pattern (XRPD pattern) as shown in any one of FIGS. 1-5.

In some aspects, the omadacycline crystalline freebase, e.g., the polymorph of omadacycline crystalline freebase as described above, is at least 90% pure, as expressed by weight of the polymorph of omadacycline crystalline freebase vs. weight of the composition (w/w %). For example, the polymorph of omadacycline crystalline freebase is at least 95% pure, at least 96% pure, at least 97% pure, at least 98% pure, at least 99% pure, at least 99.1% pure, at least 99.5% pure, or at least 99% pure. As used herein, the terms “pure” or “purity” refer to a compound that is about 90-100% pure, e.g., about 95-100% pure, 98-100% pure, or about 99-100% pure. In some embodiments, the omadacycline free base that is pure comprises less than about 10%, less than about 5%, less than about 2% or less than about 1% of impurities. The impurities may include, e.g., one or more of degradation products, oxidized products, epimers, solvents, and/or other undesirable impurities.

One measure of purity of the omadacycline crystalline freebase, e.g., the polymorph of omadacycline crystalline freebase as described above, is defined by the content of the 4-epi-isomer (β-epimer) represented by formula (5) as shown above. Thus, in some examples, the content of β-epimer in the omadacycline crystalline freebase, e.g., the polymorph of omadacycline crystalline freebase as described above, is 10% or less, e.g., 5% or less, 2% or less, 1% or less, 0.9% or less, 0.5% or less, 0.1% or less, 0.05% or less or 0.01% or less as measured by HPLC (i.e., % of total area under the curve on the HPLC trace). In one specific example, the content of β-epimer in the omadacycline crystalline freebase, e.g., the polymorph of omadacycline crystalline freebase as described above, is 0.9% or less.

### Methods of Synthesizing Omadacycline Crystalline Freebase

Omadacycline crystalline freebase, e.g., the polymorph of omadacycline crystalline freebase as described above, may be prepared by crystallizing amorphous omadacycline free base from a solvent system that comprises an organic solvent and water.

In such solvent system, the organic solvent and water may be present at a ratio ranging from about 5:95 v/v to about 95:5 v/v organic solvent:water. For example, water may be present in the solvent system in an amount of about 95% v/v (i.e., at a ratio of organic solvent:water of about 5:95 v/v), about 90% v/v (i.e., at a ratio of organic solvent:water of about 10:90 v/v), about 80% v/v (i.e., at a ratio of organic solvent:water of about 20:80 v/v), about 70% v/v (i.e., at a ratio of organic solvent:water of about 30:70 v/v), about 60% v/v (i.e., at a ratio of organic solvent:water of about 40:60 v/v), about 50% v/v (i.e., at a ratio of organic solvent:water of about 50:50 v/v), about 40% v/v (i.e., at a ratio of organic solvent:water of about 60:40 v/v), about 30% v/v (i.e., at a ratio of organic solvent:water of about 70:30 v/v), about 20% v/v (i.e., at a ratio of organic solvent:water of about 80:20 v/v), about 15% v/v (i.e., at a ratio of organic solvent:water of about 85:15 v/v), about 10% v/v (i.e., at a ratio of organic solvent:water of about 90:10 v/v), about 5% v/v (i.e., at a ratio of organic solvent:water of about 95:5 v/v), or about 1% v/v (i.e., at a ratio of organic solvent:water of about 99:1 v/v). In other examples, water may be present in the solvent system in an amount of about 1% to about 10%, about 2% to about 15%, about 5% to about 20%, or about 1% to about 15%.

The organic solvent present in a solvent system may be selected from the group consisting of a nitrile, an alcohol, a ketone and an ether. For example, the organic solvent may be selected from the group consisting of acetonitrile, acetone, isopropyl alcohol, methyl ethyl ketone, t-butyl methyl ether, ethyl acetate, toluene and tetrahydrofuran. In some examples, the organic solvent may be selected from the group consisting of acetonitrile, acetone, isopropyl alcohol and methyl ethyl ketone.

In one example, the solvent system may comprise acetone and water. The acetone and water may be present in the solvent system at a ratio of acetone:water of about 1:99 v/v to about 99:1 v/v, e.g., about 10:90 v/v to about 90:10 v/v, about 20:80 v/v to about 80:20 v/v, about 30:70 v/v to about 70:30, about 40:60 v/v to about 60:40 v/v. In one example, the acetone and water may be present in the solvent system at a ratio of acetone:water of about 50/50 v/v. In another example, the solvent system may comprise acetonitrile and water, e.g., present in the solvent system at a ratio of acetonitrile:water of about 95/5 v/v. In yet another example, the solvent system may comprise isopropyl alcohol and water, e.g., present in the solvent system at a ratio of isopropyl alcohol:water of about 95/5 v/v. In another example, the solvent system may comprise methyl ethyl ketone and water, e.g., present in the solvent system at a ratio of methyl ethyl ketone:water of about 95/5 v/v.

In some examples, prior to crystallization, omadacycline amorphous freebase may be purified from a solution comprising crude omadacycline amorphous freebase by high performance liquid chromatograph (HPLC) and/or nanofiltration as described elsewhere in this disclosure.

In some embodiments, the present invention provides omadacycline crystalline freebase prepared by the methods described above.

### Pharmaceutical Compositions Comprising Omadacycline Crystalline Freebase

The present invention also provides pharmaceutical compositions comprising omadacycline crystalline freebase. For example, a pharmaceutical composition of the present invention may comprise an effective amount of the omadacycline crystalline freebase and, optionally, a pharmaceutically acceptable carrier. The pharmaceutical composition of the invention may be administered to a subject in need thereof for treating or preventing a bacterial infection.

The language “pharmaceutically acceptable carrier” includes substances capable of being co-administered with omadacycline crystalline freebase, and which may allow both to perform their intended function, e.g., treat or prevent a bacterial infection. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical compositions may be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the omadacycline freebase.

The pharmaceutical compositions of the present invention comprising omadacycline crystalline freebase may be adapted for administration via either the oral, parenteral or topical routes. In general, omadacycline crystalline freebase is most desirably administered in effective dosages, depending upon the weight and condition of the subject being treated and the particular route of administration chosen. Variations may occur depending upon the species of the subject being treated and its individual response to the medicament, as well as on the type of pharmaceutical composition chosen and the time period and interval at which such administration is carried out.

For oral administration, omadacycline crystalline freebase may be administered in the form of a tablet or a capsule. The tablet or a capsule may comprise various excipients, e.g., an excipient selected from the group consisting of microcrystalline cellulose, sodium citrate, calcium carbonate, dicalcium phosphate and glycine. The tablet or a capsule may also comprise a disintegrant, e.g., starch (and preferably corn, potato or tapioca starch), alginic acid and certain complex silicates. The tablet or a capsule may also comprise a granulation binder, e.g., sucrose, gelatin or acacia. Additionally, lubricating agents, such as magnesium stearate, sodium lauryl sulfate and talc may also be added to a tablet or a capsule for tableting purposes.

In some examples, an oral formulation comprising omadacycline crystalline freebase comprises at least one or more of an additional ingredient, such as a diluent, a stabilizer, a glidant, a lubricant, and a disintegrant. In some examples, the diluent may be lactose or microcrystalline cellulose, or a combination of both lactose and microcrystalline cellulose. In some examples, the stabilizer may be sodium bisulfite. In some examples, the glidant may be colloidal silicon dioxide. In some examples, the lubricant may be sodium stearyl fumarate or magnesium stearate. In some examples, the disintegrant may be crospovidone.

In some embodiments, a pharmaceutical composition of the present invention intended for oral administration, e.g., a tablet or a capsule, may comprise about 10 to about 1000 mg of omadacycline crystalline freebase, e.g., about 20 to about 750 mg, about 50 to about 500 mg, about 75 to about 400 mg, about 100 to about 300 mg, about 110 to about 250 mg, about 120 to about 240 mg, about 130 to about 210 mg, about 140 to about 170 mg, or about 150 mg of omadacycline crystalline freebase.

For parenteral administration (including intraperitoneal, subcutaneous, intravenous, intradermal or intramuscular injection), the present invention also provides injectable formulations comprising omadacycline crystalline freebase. Such injectable formulations may be in the form of a dry, e.g., lyophilized, powder, that is reconstituted with a carrier, e.g., an aqueous carrier, such as water, prior to administration. In some embodiments, the injectable formulation comprising omadacycline crystalline freebase may also comprise at least one or more of an additional ingredient, such as a lyoprotectant, an antioxidant and a pH adjustment compound.

In some examples, the lyoprotectant may be a sugar, such as a sucrose. In some examples, the antioxidant may be a bisulfite compound, e.g., sodium bisulfite. In some examples, the pH adjustment compound may be an acid, e.g., a mineral acid, such as phosphoric acid, nitric acid, sulfuric acid or hydrochloric acid. The pH adjustment compound may also be a base, e.g., a mineral base, such as sodium hydroxide. In some examples, the pH adjustment compound may comprise both an acid, e.g., a mineral acid, and a base, that are added together to the injectable formulation comprising omadacycline crystalline salt in order to achieve a desired pH. In some embodiments, the desired pH is about 4.0 to about 4.5, e.g., 4.2.

In some embodiments, a pharmaceutical composition of the present invention intended for parenteral administration, e.g., an injectable formulation in lyophilized form or reconstituted with a carrier, may comprise about 5 to about 500 mg of omadacycline crystalline freebase, e.g., about 10 to about 400 mg, about 25 to about 300 mg, about 50 to about 200 mg, about 50 to about 150 mg, about 60 to about 140 mg, about 70 mg to about 130 mg, about 80 mg to about 120 mg, about 90 mg to about 110 mg, or about 100 mg of omadacycline crystalline freebase.

In some embodiments, the pharmaceutical composition comprising omadacycline crystalline freebase may be in the form of an aerosol pharmaceutical composition. Such aerosol pharmaceutical compositions may be in the form of a solution, a suspension, a powder formulation or a liposomal formulation. An aerosol pharmaceutical composition comprising omadacycline crystalline freebase may be contained in an aerosol dispenser that may, in some examples, also comprise a metered dose spray device. In some examples, the aerosol dispenser may be a nebulizer, e.g., a small-volume nebulizer (SVN), a pressurized metered-dose inhaler (pMDI) or a dry-powder inhaler (DPI). Administration of a tetracycline compound, e.g., omadacycline, or a pharmaceutically acceptable salt thereof, via an aerosol within the context of the present invention may be particularly useful for treating a pulmonary disease, e.g., a pulmonary disease associated with a bacterial infection, such as a mycobacterial infection.

In some embodiments, the pharmaceutical composition comprising omadacycline crystalline freebase may be in the form of a pharmaceutical composition adapted for topical administration. Such pharmaceutical compositions may be in a form of a gel, an ointment, a lotion or a cream, and may comprise the tetracycline compound suitably admixed in a pharmacologically inert topical carrier. The pharmacologically inert topical carriers may include water, glycerol, alcohol, propylene glycol, fatty alcohols, triglycerides, fatty acid esters, or mineral oils. Other possible topical carriers may be liquid petrolatum, isopropylpalmitate, polyethylene glycol, ethanol, polyoxyethylene monolauriate, sodium lauryl sulfate and the like. In addition, materials such as anti-oxidants, humectants, viscosity stabilizers and the like also may be added if desired.

### Methods for Treating or Preventing Bacterial Infections Using Omadacycline Crystalline Freebase

The present invention also provides methods for treating or preventing a bacterial infection that comprise administering to a subject in need thereof omadacycline crystalline freebase or a pharmaceutical composition comprising omadacycline crystalline freebase.

In some embodiments, the bacterial infection may be caused by a Gram-positive or a Gram-negative bacteria. In some embodiments, the bacterial infection may be caused by a bacteria that is resistant to other tetracycline compounds. In some examples, the bacterial infection may be caused by a bacteria of a species selected from the group consisting of K. pneumoniae, Salmonella, E. hirae, A. baumanii, B. catarrhalis, H. influenza, P. aeruginosa, E. faecium, E. coli, S. aureus and E. faecalis.

In one example, the bacterial infection is an acute bacterial skin structure infection (ABSSSI). ABSSSI may be caused by a Gram-positive or a Gram-negative bacteria, e.g., a bacteria of a species selected from the group consisting of Staphylococcus aureus (methicillin-susceptible and -resistant isolates), including cases with concurrent bacteremia, Staphylococcus lugdunensis, Streptococcus pyogenes, Streptococcus agalactiae, Streptococcus anginosus grp. (includes S. anginosus, S. intermedius, and S. constellatus), Streptococcus mitis, Enterococcus faecalis (vancomycin-susceptible isolates), Enterobacter cloacae, Klebsiella pneumoniae, Prevotella melaninogenica, and Finegoldia magna.

In another example, the bacterial infection is community-acquired bacterial pneumonia (CABP). CABP may be caused by a Gram-positive, a Gram-negative or an atypical bacteria, e.g., a bacteria of a species selected from the group consisting of Streptococcus pneumoniae (penicillin-susceptible and -resistant isolates, macrolide-resistant isolates), including cases with concurrent bacteremia, Staphylococcus aureus (methicillin-susceptible isolates), Haemophilus influenzae (beta-lactamase negative and positive isolates), Haemophilus parainfluenzae, Klebsiella pneumoniae, Legionella pneumophila, Mycoplasma pneumoniae, and Chlamydophila pneumoniae.

In another example, the bacterial infection may be caused by C. difficile.

In another example, the bacterial infection may be caused by a mycobacteria, e.g., mycobacteria that belongs to a mycobacterial species described in U.S. Patent Application No. 62/726,738, U.S. Patent Application No. 62/731,410, U.S. Patent Application No. 62/746,039 and U.S. Patent Application No. 62/760,131, the entire contents of each of which are hereby incorporated herein by reference.

In yet another example, the bacterial infection is a urinary tract infection (UTI).

The term “treating” or “treatment” refers to the amelioration or diminishment of one or more symptoms of the disorder, e.g., a bacterial infection, to be treated.

The term “prophylaxis”, “prevent”, or “prevention” means to prevent or reduce the risk of a bacterial infection.

The term “resistance” or “resistant” refers to the antibiotic/organism standards as defined by the Clinical and Laboratories Standards Institute (CLSI) and/or the Food and Drug Administration (FDA).

The term “subject” includes animals which are subject to a bacterial infection. Examples of subjects include animals such as farm animals (e.g., cows, pigs, horses, goats, rabbits, sheep, chickens, etc.), lab animals (mice, rats, monkeys, chimpanzees, etc.), pets (e.g., dogs, cats, ferrets, hamsters, etc.), birds (e.g., chickens, turkeys, ducks, geese, crows, ravens, sparrows, etc.), primates (e.g., monkeys, gorillas, chimpanzees, bonobos, and humans), and other animals (e.g., squirrels, raccoons, mice, rats, etc.). In one embodiment, the subject is a mouse or rat. In one embodiment, the subject is a cow, a pig, or a chicken. In one embodiment, the subject is a human.

The term “effective amount” includes the amount of omadacycline crystalline freebase needed to treat or prevent a bacterial infection. For example, an effective amount describes an efficacious level sufficient to achieve the desired therapeutic effect through the killing of bacteria and/or inhibition of bacterial growth. In one embodiment, the effective amount is sufficient to eradicate the bacterium or bacteria causing the infection.

### Administration of Omadacycline Crystalline Freebase

Omadacycline crystalline freebase may be administered to a subject in need thereof alone or as a part of a pharmaceutical composition. Any exemplary pharmaceutical composition comprising omadacycline crystalline freebase may comprise an effective amount of omadacycline crystalline freebase and, optionally, a pharmaceutically acceptable carrier.

The language “pharmaceutically acceptable carrier” includes substances capable of being co-administered with omadacycline crystalline freebase and which may allow both to perform their intended function, e.g., treat or prevent a bacterial infection. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical compositions may be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with omadacycline crystalline freebase.

The pharmaceutical compositions that may be used in the methods of the present invention may be adapted for administration via either the oral, parenteral, or topical routes. In some examples, the pharmaceutical compositions that may be used in the methods of the present invention may also be adapted for delivery via aerosol. In general, omadacycline crystalline freebase is most desirably administered in effective dosages, depending upon the weight and condition of the subject being treated and the particular route of administration chosen. Variations may occur depending upon the species of the subject being treated and its individual response to the omadacycline crystalline freebase, as well as on the type of pharmaceutical composition chosen and the time period and interval at which such administration is carried out.

For oral administration, omadacycline crystalline freebase may be administered in the form of a tablet or a capsule. The tablet or a capsule may comprise various excipients, e.g., an excipient selected from the group consisting of microcrystalline cellulose, sodium citrate, calcium carbonate, dicalcium phosphate and glycine. The tablet or a capsule may also comprise a disintegrant, e.g., starch (and preferably corn, potato or tapioca starch), alginic acid and certain complex silicates. The tablet or a capsule may also comprise a granulation binder, e.g., sucrose, gelatin or acacia. Additionally, lubricating agents, such as magnesium stearate, sodium lauryl sulfate and talc may also be added to a tablet or a capsule for tableting purposes.

For parenteral administration (including intraperitoneal, subcutaneous, intravenous, intradermal or intramuscular injection), the present invention also provides injectable formulations comprising omadacycline crystalline freebase. Such injectable formulations may be in the form of a dry, e.g., lyophilized, powder, that is reconstituted with a carrier, e.g., an aqueous carrier, such as water, prior to administration. In some embodiments, the injectable formulation may also comprise at least one or more of an additional ingredient, such as a lyoprotectant, an antioxidant and a pH adjustment compound.

Certain pharmaceutical compositions comprising omadacycline crystalline freebase that may be suitable for use in the methods of the present invention, are described, e.g., in U.S. Pat. No. 9,315,475, the entire contents of which are hereby incorporated herein by reference.

In the methods of the present invention, omadacycline crystalline freebase may also be administered to a subject by an aerosol. An aerosol pharmaceutical composition comprising omadacycline crystalline freebase may be in the form of a solution, a suspension, a powder formulation or a liposomal formulation. An aerosol pharmaceutical composition comprising omadacycline crystalline freebase may be contained in an aerosol dispenser that may, in some examples, also comprise a metered dose spray device. In some examples, the aerosol dispenser may be a nebulizer, e.g., a small-volume nebulizer (SVN), a pressurized metered-dose inhaler (pMDI) or a dry-powder inhaler (DPI).

For topical administration, omadacycline crystalline freebase may also be administered to a subject as a part of a pharmaceutical composition adapted for topical administration. Such compositions may be in a form of a gel, an ointment, a lotion or a cream, and may comprise the tetracycline compound suitably admixed in a pharmacologically inert topical carrier. The pharmacologically inert topical carriers may include water, glycerol, alcohol, propylene glycol, fatty alcohols, triglycerides, fatty acid esters, or mineral oils. Other possible topical carriers may be liquid petrolatum, isopropylpalmitate, polyethylene glycol, ethanol, polyoxyethylene monolauriate, sodium lauryl sulfate and the like. In addition, materials such as anti-oxidants, humectants, viscosity stabilizers and the like also may be added if desired.

Omadacycline crystalline freebase may be administered to a subject at a dose, e.g., daily dose, of from about 100 to about 200 mg, from about 100 to about 300 mg, from about 100 to 400 mg, from about 100 to about 500 mg, from about 100 to about 600 mg, from about 200 to about 500 mg, or from about 300 to about 600 mg of omadacycline crystalline freebase. In a further example, omadacycline crystalline freebase may be administered orally. In a further example, omadacycline crystalline freebase may be administered intravenously.

In some aspects, omadacycline crystalline freebase may be administered to a subject at a dose of about 50 to about 150 mg, about 50 to about 400 mg, about 50 to about 300 mg, about 50 to about 200 mg, about 100 to about 300 mg or about 200 to about 300 mg, or about 100 mg. For example, omadacycline crystalline freebase may be administered to a subject at a dose, e.g., a daily dose, of about 100 mg, about 150 mg, about 200 mg, about 250 mg or about 300 mg. In one embodiment, the dose is an intravenous dose.

In some aspects, omadacycline crystalline freebase may be administered to a subject at a dose of from about 50 to about 800 mg, about 100 to about 700 mg, about 250 to about 600 mg, about 300 to about 500 mg, about 100 to about 400 mg, about 100 to about 600 mg, or about 300 mg. For example, omadacycline crystalline freebase may be administered at a dose of about 300 mg, about 450 mg or about 600 mg. In one embodiment, the dose is an oral dose.

In an embodiment, omadacycline crystalline freebase may be administered intravenously at a dose of about 100 mg, about 200 mg, or about 300 mg. In another embodiment, omadacycline crystalline freebase may be administered orally at the dose of about 300 mg, about 600 mg, or about 900 mg.

In some examples, omadacycline crystalline freebase may be administered as an aerosol dose, e.g., delivered using an aerosol dispenser. In some examples, the aerosol dispenser may comprise a dose of omadacycline crystalline freebase of about 1 to about 2000 mg, e.g., about 1 to about 500 mg, about 25 to about 300 mg, about 50 to about 400 mg, about 100 to about 500 mg, about 200 to about 800 mg, about 500 mg to about 1000 mg, about 10 mg to about 200 mg or about 300 mg to about 700 mg. In some examples, the aerosol dispenser may comprise a dose of omadacycline crystalline freebase of about 1 mg, about 5 mg, about 10 mg, about 30 mg, about 50 mg, about 80 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg or about 1000 mg.

In some examples, omadacycline crystalline freebase may be administered topically, e.g., by applying to an affected area pharmaceutical composition adapted for topical administration comprising omadacycline crystalline freebase. For example, the pharmaceutical composition adapted for topical administration may be in the form of a solution and comprise omadacycline crystalline freebase at a concentration of about 0.01% to about 20% w/v based on the volume of the composition, e.g., about 0.01% to about 10% w/v, about 0.1% to about 20% w/v, about 0.5% to about 5% w/v, about 1% to about 10% w/v or about 5% to about 20% w/v. For example, the pharmaceutical composition adapted for topical administration may comprise omadacycline crystalline freebase at a concentration of about 0.01% w/v, about 0.05% w/v, about 0.1% w/v, about 0.5% w/v, about 1% w/v, about 5% w/v, about 10% w/v, about 15% w/v or about 20% w/v.

In another example, the pharmaceutical composition adapted for topical administration may comprise omadacycline crystalline freebase at a concentration of about 0.01% to about 20% w/w based on the volume of the composition, e.g., about 0.01% to about 10% w/w, about 0.1% to about 20% w/w, about 0.5% to about 5% w/w, about 1% to about 10% w/w or about 5% to about 20% w/w. For example, the pharmaceutical composition adapted for topical administration may comprise omadacycline crystalline freebase at a concentration of about 0.01% w/w, about 0.05% w/w, about 0.1% w/w, about 0.5% w/w, about 1% w/w, about 5% w/w, about 10% w/w, about 15% w/w or about 20% w/w.

In some examples, omadacycline crystalline freebase may be administered at the doses as described above at least once daily, e.g., once daily, twice daily, three times daily or four times daily. In further examples, omadacycline crystalline freebase may be administered to a subject twice daily. In one specific example, omadacycline crystalline freebase may be administered orally to a subject twice daily.

It should be understood that administration of dose ranges comprising the above listed doses is also included in the present invention. For example, any of the above doses may be a lower part or an upper part of a dose range that is included in the methods of the present invention. Even further, it should be understood that all lists or collections of numerical values used throughout the present application also are intended to include ranges of the numerical values wherein any of the listed numerical values can be the lower part or upper part of a range. These ranges are intended to be included in the present invention.

In one embodiment, an oral dose of omadacycline crystalline freebase may be 3 times larger than an intravenous dose of omadacycline crystalline freebase.

It will be understood that for all listed embodiments, the dose of omadacycline crystalline freebase is also an effective amount of omadacycline crystalline freebase.

In one embodiment, the effective amount of omadacycline crystalline freebase, when administered orally, may be from about 100 to about 1000 mg of omadacycline crystalline freebase, e.g., from about 200 to about 750 mg, about 100 to about 500 mg, about 200 to about 600 or about 400 to about 600. In a further example, the effective amount of omadacycline crystalline freebase, when administered orally, may be about 300 mg, about 450 mg or about 600 mg of the tetracycline compound.

In another embodiment, the effective amount of omadacycline crystalline freebase, when administered intravenously, may be from about 50 to about 500 mg omadacycline, e.g., about 50 to about 400 mg, about 100 to about 300 mg or about 50 to about 200 mg. For example, the effective amount of omadacycline crystalline freebase, when administered intravenously, may be about 100 mg, about 150 mg, about 200 mg, about 250 mg or about 300 mg.

In some examples, omadacycline crystalline freebase may be administered in the context of the present invention via either the oral, parenteral, systemic, topical routes, or via aerosol delivery. In general, omadacycline crystalline freebase is most desirably administered in an effective dosage, depending upon the weight and condition of the subject being treated and the particular route of administration chosen. Variations may occur depending upon the species of the subject being treated and its individual response to the medicament, as well as on the type of pharmaceutical formulation chosen and the time period and interval at which such administration is carried out.

In some embodiments, omadacycline crystalline freebase may be administered for at least 3 days, at least 7 days, at least 14 days, at least 21 days, at least 30 days, at least 60 days, at least 5 weeks, at least 10 weeks, at least 15 weeks, at least 20 weeks, at least 30 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months. For example, the administration of omadacycline crystalline freebase may last from 3 days to 7 days, from 3 days to 14 days, from 3 days to 21 days, from 3 days to 30 days, from 3 days to 60 days, from 7 days to 14 days, from 7 days to 21 days, from 7 days to 30 days, from 7 days to 60 days, from 14 days to 21 days, from 14 days to 30 days, from 14 days to 60 days, from 21 days to 30 days, from 21 days to 60 days, from 30 days to 60 days, from 1 week to 5 weeks, from 3 weeks to 10 weeks, from 5 weeks to 20 weeks, from 10 weeks to 30 weeks, from 20 weeks to 35 weeks, from 1 week to 1 month, from 2 weeks to 2 months, from 1 month to 3 months, from 1 month to 6 months, from 1 month to 9 months, from 3 months to 12 months, from 6 months to 12 months, from 9 months to 12 months, from 9 months to 16 months, from 12 months to 18 months, from 14 months to 24 months, from 12 months to 24 months, or for 24 months or longer.

For example, omadacycline crystalline freebase may be administered for 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months or 24 months. In other examples, omadacycline crystalline freebase may be administered for longer than 24 months, e.g., 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 month, or longer than 48 months.

In some embodiments, administration of omadacycline crystalline freebase to a subject may comprise administering one or more loading doses of the omadacycline crystalline freebase, followed by one or more maintenance doses of the omadacycline crystalline freebase. In some embodiments, the one or more loading dose of omadacycline crystalline freebase may be greater than the one or more maintenance dose of omadacycline crystalline freebase. For example, the loading dose may be about 450 mg daily dose, e.g., a daily oral dose, while the maintenance dose may be about 300 mg daily dose, e.g., a daily oral dose. In another example, the loading dose may be about 200 mg daily dose, e.g., a daily intravenous dose, while the maintenance dose may be about 100 mg daily dose, e.g., a daily intravenous dose, or a 300 mg daily dose, e.g., a daily oral dose.

The loading dose of omadacycline crystalline freebase and the maintenance dose of omadacycline crystalline freebase may be administered via the same route or different routes. For example, the loading dose(s) may be administered intravenously and the maintenance dose may be administered orally. In other embodiments, both the loading dose(s) and the maintenance doses may be administered orally, or both the loading dose(s) and the maintenance dose may be administered intravenously.

In some examples, the loading dose of omadacycline crystalline freebase may be an oral dose or an intravenous dose administered twice daily, and the maintenance dose may be an oral dose or an intravenous dose administered once daily. For example, omadacycline crystalline freebase may be administered as an intravenous loading dose of 100 mg twice daily, followed by an intravenous maintenance dose of 100 mg once daily. In another example, omadacycline crystalline freebase may be administered as an intravenous loading dose of 100 mg twice daily, followed by an oral maintenance dose of 300 mg once daily. In yet another example, omadacycline crystalline freebase may be administered as an oral loading dose of 300 mg twice daily, followed by an oral maintenance dose of 300 mg once daily.

In another example, administration of omadacycline crystalline freebase may not comprise administration of one or more loading doses of the omadacycline crystalline freebase. Thus, in some examples, omadacycline crystalline freebase may be administered to a subject at the same dose throughout the treatment period. For example, omadacycline crystalline freebase may be administered to the subject at an intravenous dose of about 100 mg, about 200 mg or about 300 mg. The intravenous dose may be administered to the subject once or twice daily throughout the treatment. In another example, omadacycline crystalline freebase may be administered to a subject at an oral dose of about 300 mg, about 450 mg or about 600 mg. The oral dose may be administered to the subject once daily throughout the treatment period.

In some examples, omadacycline crystalline freebase may be administered to a subject alone or in combination with at least one additional therapeutic agent. The language “in combination with” a therapeutic agent is intended to include simultaneous administration of omadacycline crystalline freebase and the therapeutic agent; administration of omadacycline crystalline freebase first, followed by the therapeutic agent; and administration of the therapeutic agent first, followed by omadacycline crystalline freebase. In one example, the therapeutic agent is an antibiotic.

The term “about” refers to a range of values which can be 15%, 10%, 8%, 5%, 3%, 2%, 1%, or 0.5% more or less than the specified value. For example, “about 10%” can be from 8.5% to 11.5%. In one embodiment, the term “about” refers to a range of values which are 5% more or less than the specified value. In another embodiment, the term “about” refers to a range of values which are 2% more or less than the specified value. In another embodiment, the term “about” refers to a range of values which are 1% more or less than the specified value.

It is to be understood that wherever values and ranges are provided herein, e.g., in ages of subject populations, dosages, and time durations, etc., all values and ranges encompassed by these values and ranges, are meant to be encompassed within the scope of the present invention. Moreover, all values in these values and ranges may also be the upper or lower limits of a range.

### Methods of Purifying Omadacycline Freebase

The present invention also provides methods of purifying omadacycline freebase, e.g., crude omadacycline freebase. The methods of purifying the omadacycline freebase involve, in some examples, the step of subjecting a solution comprising crude omadacycline freebase to purification by high performance liquid chromatography (HPLC), e.g., preparative HPLC, thereby obtaining a solution comprising HPLC-purified omadacycline freebase. In some embodiments, the method of purifying omadacycline freebase may also comprise, subsequent to the HPLC purification, concentrating the solution comprising HPLC-purified omadacycline freebase using nanofiltration, thereby obtaining a concentrated solution comprising HPLC-purified omadacycline freebase. In some embodiments, the method of purifying omadacycline freebase may also comprise, subsequent to the nanofiltration step, crystallizing omadacycline freebase from the concentrated solution comprising HPLC-purified omadacycline freebase, thereby producing omadacycline crystalline freebase, e.g., the polymorph of omadacycline crystalline freebase as described above.

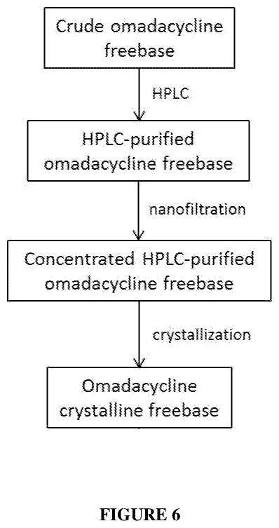

Methods of purifying omadacycline freebase, e.g., crude omadacycline freebase, provided by the present invention allow achieving higher throughput and productivity of omadacycline purification than previous methods of purifying omadacycline (as described, e.g., in U.S. Pat. No. 9,434,680). Specifically, in some embodiments, methods of purifying omadacycline provided by the present invention yield purified omadacycline freebase which comprises lower levels of impurities, e.g., the 4-epi-isomer (β-epimer) represented by formula (5) as shown above, than previous methods. In some embodiments, methods of purifying omadacycline provided by the present invention are characterized by significantly shorter processing times than previous methods of omadacyline purification. The term “processing time”, as used herein, refers to the amount of time required to produce 1 kilogram of omadacycline crystalline freebase starting from crude omadacycline freebase. In some examples, the term “processing time” is the time required to produce 1 kilogram of omadacycline crystalline freebase in accordance with the procedure as illustrated in FIG. 6, i.e., the procedure that includes the steps of HPLC, nanofiltration and crystallization. In some examples, the term “processing time” does not include the time required for drying omadacycline crystalline freebase after crystallization.

For example, as compared with previous method, the present methods allow to reduce processing time by at least 2-fold, e.g., at least about 3-fold, at least about 4-fold, at least about 5-fold, at least about 6-fold, at least about 7-fold, at least about 8-fold, at least about 9-fold, at least about 10-fold, at least about 11-fold or at least about 12-fold as compared with the processing time required with previous methods. In one specific embodiment, the present methods allow to reduce processing time by at least about 12-fold, i.e., from about 73.9 hours achieved with the previous method to about 6.1 hours.

### High Performance Liquid Chromatography

In some examples, the method of purifying omadacycline freebase comprises the step of subjecting a solution comprising crude omadacycline freebase to purification by high performance liquid chromatography (HPLC), e.g., preparative HPLC, thereby obtaining a solution comprising HPLC-purified omadacycline freebase. Purification by HPLC of crude omadacycline freebase allows removal of impurities, e.g., compounds (3), (4) and (5) as described above, as well as synthesis by-products. The solution comprising crude omadacycline freebase may be obtained by any method known in the art for preparing crude omadacycline freebase, e.g., by a procedure illustrated in Scheme 1 above or by methods described, e.g., in U.S. Pat. Nos. 9,434,680, 9,522,872, or U.S. Pat. No. 8,383,610, the entire contents of each of which are incorporated herein by reference.

In some embodiments, the HPLC purification comprises the use of stationary phase, e.g., reversed phase (RP). The stationary phase that may be used in the context of the present invention may be any stationary phase providing adequate purification of crude omadacycline freebase. In some examples, the RP stationary phase may be a C18 stationary phase. In some examples, the RP stationary phase may have a pore size of about 50 Å to about 200 Å, e.g., about 50 Å to about 90 Å, about 80 Å to about 150 Å, about 120 Å to about 200 Å, or about 100 Å. In some examples, the RP stationary phase may have a particle size of from about 0.5 μm to about 20 μm, e.g., about 0.5 μm to about 5 μm, about 2 μm to about 15 μm, about 5 μm to about 20 inn, or about 10 inn. In one example, the RP stationary phase may be a C18 phase having a pore size of about 100 Å and a particle size of about 10 inn. In one embodiment, the RP stationary phase may be Luna® 10 μm PREP C18(2) 100 Å, LC Column 250×21.2 mm, AXIA™ Packed, Ea from Phenomenex. In another embodiment, the RP stationary phase may be Synergi Polar RP10 μ80 Å.

In some embodiments, the HPLC purification comprises the use of mobile phase, e.g., elution buffer, such as elution buffer A and elution buffer B. The elution buffer A may comprise a mixture of water and an organic solvent, such as methanol, ethanol, methylene chloride or acetonitrile. In a specific embodiment, buffer A comprises water and acetonitrile. The elution buffer A may also comprise, in some examples, an inorganic salt, such as a phosphate salt (e.g., potassium phosphate, dibasic potassium phosphate, sodium phosphate, dibasic sodium phosphate or ammonium phosphate), an acetate salt (e.g., ammonium acetate, potassium acetate or sodium acetate) or a formate salt (e.g., sodium formate or potassium formate). The elution buffer may B may comprise an organic solvent, such as methanol, ethanol, methylene chloride or acetonitrile. In a specific embodiment, the elution buffer B comprises acetonitrile.

In some embodiments, elution buffer A and/or elution buffer B may also comprise a modifier selected from the group consisting of a strong acid which is not methyl sulfonic acid (e.g., hydrochloric acid), a weak acid and an organic amine. In other embodiments, a modifier selected from the group consisting of a weak acid and an organic amine may be added to the solution comprising crude omadacycline freebase prior to HPLC purification.

As used herein, the term “modifier” refers to a chemical agent which, when added to elution buffer A, and/or elution buffer B, and/or solution comprising crude omadacycline freebase, facilitates purification of the crude omadacycline freebase by HPLC.

As used herein, the term “weak acid” refers to an acid that does not dissociate completely when dissolved in a solution, e.g., in an aqueous solution. In some embodiments, a weak acid is an acid having a pKa of between about 3.0 and about 6.0.

In some embodiments, the modifier may be a weak acid. Non-limiting examples of a weak acid that may be used as a modifier in the context of the present invention may include oxalic acid, methanesulfonic acid, trifluoracetic acid, sulfurous acid, phosphoric acid, nitrous acid, hydrofluoric acid, benzoic acid, acetic acid and formic acid. For example, the weak acid may be selected from the group consisting of oxalic acid, methanesulfonic acid, trifluoracetic acid, benzoic acid, acetic acid and formic acid. In one specific example, the weak acid may be acetic acid.

In other embodiments, the modifier may be an organic amine. Non-limiting examples of the organic amine that may be used as a modifier in the context of the present invention include 3-ethanolamine, diethylamine and trimethylamine.

In one specific embodiment, the modifier is a weak acid, e.g., acetic acid. In a further example, the weak acid, e.g., acetic acid, is added to elution buffer A. In another further example, the weak acid, e.g., acetic acid, is added to elution buffer B. In yet another further example, the weak acid, e.g., acetic acid, is added to a solution comprising crude omadacycline freebase prior to HPLC purification.

Without wishing to be bound by a specific theory, it is believed that certain modifiers, such as a weak acid, e.g., acetic acid, when added to elution buffer A, and/or elution buffer B, and/or solution comprising crude omadacycline freebase prior to HPLC purification, allows quicker and more efficient purification of omadacycline freebase by HPLC as compared to the previous HPLC based purification methods used for omadacycline. The previous method of omadacycline purification, e.g., as described in U.S. Pat. No. 8,946,196, the entire contents of which are incorporated herein by reference, utilized a strong acid, i.e., methane sulfonic acid, as a modifier of the mobile phase. Without wishing to be bound by a specific theory, it is believed that adding a weak acid, e.g., acetic acid, to elution buffer A, and/or elution buffer B, and/or solution comprising crude omadacycline freebase prior to HPLC purification, produces a well-defined HPLC peak corresponding to omadacycline freebase, e.g., an HPLC peak with sharp borders. This, in turn, allows collecting a single HPLC fraction or a few HPLC fractions comprising omadacycline freebase instead of many HPLC fractions, as was required with the previous purification method. This, in turn, allows to load more material, i.e., crude omadacycline freebase, onto the HPLC column and to collect HPLC fractions in a much shorter time than the previous purification method while also using significantly smaller amounts of solvent for HPLC. Furthermore, this also allows to achieve a better purification effect and/or recovery of the product, i.e., omadacycline freebase, as compared to the previous purification method. This makes the HPLC purification method of the present invention particularly well suited for purifying large quantities of omadacycline freebase.

One measure of purity of the omadacycline crystalline freebase, e.g., the polymorph of omadacycline crystalline freebase as described above, is defined by the content of the 4-epi-isomer (β-epimer) represented by formula (5) as shown above. In some examples, the purification of crude omadacycline freebase by HPLC, e.g., preparative HPLC, results in removal of significant amounts of the β-epimer. For example, the amount of the β-epimer present in the solution comprising HPLC-purified omadacycline freebase is at least 50%, e.g., at least 100%, at least 150%, at least 200%, at least 250%, at least 300%, at least 350%, at least 400%, at least 450%, at least 500%, at least 600%, at least 650%, at least 700%, at least 750%, at least 800%, at least 850%, at least 900%, at least 950% or at least 1000% lower, as measured by % area under the curve of the HPLC trace, than the amount of the β-epimer present in the solution comprising crude omadacycline freebase. For example, in Example 1, Table 2 described herein, the % content of β-epimer was reduced from about 11.13% β-epimer present in the solution of crude omadacycline freebase to about 1.71% β-epimer present in the solution of HPLC-purified omadacycline freebase.

### Nanofiltration

In some embodiments, the HPLC fractions containing HPLC-purified omadacycline freebase may be concentrated using nanofiltration, producing concentrated solution of HPLC-purified omadacycline freebase. In some embodiments, following nanofiltration, the concentrated solution of HPLC-purified omadacycline freebase may be extracted with a solvent, e.g., dichloromechane (DCM). In other embodiments, the concentrated solution of HPLC-purified omadacycline freebase is not extracted with a solvent, e.g., DCM, and is used directly for crystallizing omadacycline freebase, as described below.

In contrast, the previous method of omadacycline purification, e.g., as described in U.S. Pat. No. 8,946,196, did not utilize nanofiltration and only utilized extraction with a solvent, e.g., DCM. Concentrating HPLC fraction containing HPLC-purified omadacycline freebase by nanofiltration in accordance with the methods of the present invention, allows to significantly reduce the amount of solvent required for the extraction carried out after the nanofiltration step. For example, as described in Example 3 herein, with the use of nanofiltration prior to extraction with DCM, 892 mL of DCM was required to produce a concentrated solution comprising 71.04 grams of HPLC-purified omadacycline freebase. In comparison, approximately 43.97 L of DCM, or approximately 49 times more DCM, would be required to produce a concentrated solution comprising the same amount of HPLC-purified omadacycline freebase without nanofiltration. Accordingly, in exemplary embodiments of the present invention that comprise extraction of HPLC-purified omadacycline freebase with DCM, nanofiltration allows to reduce the amount of a solvent, e.g., DCM, required for extraction by at least about 5-fold, at least about 10-fold, at least about 15-fold, at least about 20-fold, at least about 25-fold, at least about 30-fold, at least about 35-fold, at least about 40-fold, at least about 45 fold, or about 50-fold. In some embodiments, nanofiltration allows to reduce the amount of a solvent, e.g., DCM, required for extraction of HPLC-purified omadacycline freebase by about 5- to about 10-fold, about 10- to about 20-fold, about 15- to about 30-fold, about 20- to about 40-fold, or about 25- to about 50 fold.

In some examples, nanofiltration to concentrate HPLC fractions containing HPLC-purified omadacycline freebase is carried out in the absence of subsequent extraction with a solvent, e.g., DCM. In these examples, the concentrated solution comprising HPLC-purified omadacycline freebase obtained after nanofiltration may be used directly for crystallizing omadacycline freebase, as described below. Therefore, in these examples, the use of nanofiltration eliminates the need for a solvent.

Reducing volume of a solvent, e.g., DCM, or eliminating the use of a solvent, e.g., DCM, for concentrating HPLC-purified omadacycline freebase, reduces the cost of the purification procedure and also eliminates the requirement for disposal of large volumes of chlorinated waste. Eliminating the use of a solvent, e.g., DCM, for concentrating HPLC-purified omadacycline freebase also eliminates the requirement for an evaporation step to remove DCM from the solution comprising HPLC-purified omadacycline freebase. The evaporation step may cause degradation of omadacycline freebase, and its elimination leads to increased recovery of omadacycline crystalline freebase.

Nanofiltration includes filtering a solution comprising HPLC-purified omadacycline freebase through a membrane. During nanofiltration, a portion of the solution being filtered passes through the membrane, forming a filtrate, while the solution remaining on top of the membrane is a retentate. In the methods of the present invention, a large portion of omadacycline freebase remains in the retentate, forming a concentrated solution comprising HPLC-purified omadacycline freebase. Therefore, in some embodiments of the present invention, the step of nanofiltration may further comprise collecting the retentate.

Membranes suitable for nanofiltration in accordance with the methods of the present invention step may include any membrane that allows retention of omadacycline freebase while allowing a solvent to pass through the membrane. In some examples, the nanofiltration membrane may have a molecular weight cut-off (MWCO) ranging from about 150 to about 500 Daltons, e.g., from about 150 to about 300 Daltons, about 200 to about 400 Daltons or about 300 to about 500 Daltons.

In some embodiments, methods of the present invention may further comprise adding an antioxidant to the solution comprising HPLC-purified omadacycline freebase prior to nanofiltration. For example, the antioxidant may be added in an amount sufficient to achieve a concentration of the antioxidant in the solution comprising HPLC-purified omadacycline freebase of about 0.01% to about 0.5% w/v of the antioxidant.

The antioxidant that may be added to the solution comprising the HPLC-purified omadacycline freebase may be selected from the group consisting of ascorbic acid (vitamin C), glutathione, lipoic acid, a carotene, α-tocopherol (vitamin E), ubiquinol (coenzyme Q), desferoxamine and a salt of bisulfite, e.g., sodium bisulfite.

The nanofiltration process results in a retentate which is a concentrated solution comprising HPLC-purified omadacycline freebase. In some examples, the concentration of omadacycline in the retentate is at least about 2 times greater, e.g., at least about 4 times greater, at least about 5 times greater, at least about 8 times greater, at least about 10 times greater, at least about 20 times greater or at least about 50 times greater, than the concentration of omadacycline in the solution comprising HPLC-purified omadacycline freebase, i.e., in fractions collected as a result of the HPLC purification.

### Crystallization

In some embodiments, the method of purifying omadacycline freebase may also comprise, subsequent to nanofiltration and extraction with a solvent, or subsequent to nanofiltration without extraction with a solvent, crystallizing omadacycline freebase, thereby producing omadacycline crystalline freebase. Crystallization of omadacycline freebase to produce, e.g., a polymorph of omadacycline crystalline freebase as described above, may be carried out according to the methods as described hereinabove.

In embodiments where omadacycline freebase is crystallized from a concentrated solution following nanofiltration without extraction with a solvent, e.g., DCM, a solvent that may be used for crystallization may be selected from the group consisting of: a mixture of acetone and water; a mixture of acetonitrile and water; and a mixture of isopropanol and water. In one example, the solvent that may be used for crystallization may be a mixture of acetone and water.

In embodiments where omadacycline freebase is precipitated from a concentrated solution following nanofiltration and extraction with a solvent, e.g., DCM, heptane and methyl tert-butyl ether (MTBE) may be used to precipitate an amorphous freebase from the DCM concentrate.

In some aspects, the omadacycline crystalline freebase produced as a result of crystallization, e.g., the polymorph of omadacycline crystalline freebase as described above, is at least 90% pure as measured by % w/w, e.g., at least, 95% pure, at least 96% pure, at least 97% pure, at least 98% pure, at least 99% pure, at least 99.1% pure, at least 99.5% pure, or at least 99% pure. As used herein, the terms “pure” or “purity” refer to a compound that is about 90-100% pure as measured by % w/w, e.g., about 95-100% pure, 98-100% pure, or about 99-100% pure. In some embodiments, the omadacycline free base that is pure comprises less than about 10%, less than about 5%, less than about 2% or less than about 1% of impurities. The impurities may include, e.g., one or more of degradation products, oxidized products, epimers, solvents, and/or other undesirable impurities.

Crystallization of omadacycline freebase from the concentrated solution comprising HPLC-purified omadacycline freebase results in further purification of omadacycline freebase, as measured, e.g., by the β-epimer content. For example, the β-epimer content in the omadacycline crystalline freebase may be reduced by at least about 50%, e.g., at least about 60%, at least about 70%, at least about 80%, or at least about 90%, as compared to the β-epimer content the concentrated solution comprising HPLC-purified omadacycline freebase. The % content of the β-epimer is measured as % total peak area corresponding to the β-epimer relative to the total area under the curve of an HPLC trace.

In some examples, the content of β-epimer in the omadacycline crystalline freebase, e.g., the polymorph of omadacycline crystalline freebase as described above, is 10% or less, e.g., 5% or less, 2% or less, 1% or less, 0.9% or less, 0.5% or less, 0.1% or less, 0.05% or less or 0.01% or less. In one specific example, the content of β-epimer in the omadacycline crystalline freebase, e.g., the polymorph of omadacycline crystalline freebase as described above, is 0.9% or less. The % content of the β-epimer is measured as % total peak area corresponding to the β-epimer relative to the total area under the curve of an HPLC trace.

The methods of purifying omadacycline freebase provided by the present invention provide advantages over the previous methods of purifying omadacycline (as described, e.g., in U.S. Pat. No. 9,434,680). The methods of purifying omadacycline freebase provided by the present invention allow achieving higher throughput and productivity of omadacycline purification as compared to the previous methods.

Table 1 below provides a comparison of the previous method of omadacycline purification and the method of the present invention. Specifically, Table 1 compares the amount of material loaded on HPLC column, calculated % recovery and the total processing time for the previous method and the present method.

TABLE 1
Results of HPLC purification using the
present method and the previous method.

| | Amount of crude | | Processing Time |
|---|---|---|---|
| | omadacycline material | Calculated | (h/kg of pure, |
| | loaded onto HPLC column | Recovery | corrected for |
| Method | (g/kg resin) | (%) | the assay) |
| Present | 122 (78 corrected for | 66.65 | 6.1 |
| | the assay) | | |
| Previous | 7.2 (4.1 corrected for | 59.41 | 73.9 |
| | the assay) | | |

The amount of crude omadacycline material loaded onto HPLC column that is shown in parentheses is the amount corrected for the assay, i.e., corrected for the actual amount of omadacycline present in the loaded material. This number reflects the actual amount of omadacycline present in the crude omadacycline material loaded onto an HPLC column. The amount of crude omadacycline material corrected for the assay may be determined by comparing HPLC signal produced by a known amount of crude omadacycline material to a calibration curve generated using an omadacycline standard of a known high purity.

The calculated % recovery may be determined by dividing the amount of crystalline omadacycline freebase obtained after purification, corrected for the assay by the amount of crude omadacycline loaded onto an HPLC column, corrected for the assay and multiplying by 100%.

Table 1 demonstrates that the present method of purification of omadacycline freebase allows loading of significantly greater amounts of crude omadacycline onto an HPLC column, thereby significantly reducing the amount of resin required for HPLC purification. The present method of purification of omadacycline freebase also results in higher calculated recovery and significantly reduces the processing time required to produce 1 kilogram of pure omadacycline freebase from about 73.9 hours to about 6.1 hours. While providing the above benefits as compared to previous methods of purification of omadacycline, the method of purification of omadacycline freebase of the present invention provides omadacycline freebase that is at least as pure as omadacycline obtained using previous methods. In some embodiments, the methods of omadacycline purification of the present invention provide omadacycline freebase that is at least 90% pure as measured by % w/w, e.g., at least 95% pure, at least 96% pure, at least 97% pure, at least 98% pure, at least 99% pure, at least 99.1% pure, at least 99.5% pure, or at least 99% pure.

These characteristics make the HPLC purification method of the present invention particularly well suited for purifying large quantities of omadacycline freebase.

### Methods of Synthesizing Tosylate Salt of Omadacycline

The present invention also provides methods of preparing a tosylate salt of omadacycline from omadacycline crystalline freebase. For example, according to methods of the present invention, tosylate salt of omadacycline may be prepared by reacting omadacycline crystalline freebase in a tosylation reaction, thereby obtaining a tosylate salt of omadacycline. In some examples, the tosylate salt of omadacycline is a crystalline tosylate salt of omadacycline, e.g., Form 1 polymorph, Form 2 polymorph or Form 3 polymorph of the crystalline tosylate salt of omadacycline as described in U.S. Pat. No. 8,383,610, the entire contents of which are incorporated herein by reference.

Methods of synthesizing tosylate salt of omadacycline, e.g., crystalline tosylate salts, such as Form 1 polymorph, Form 2 polymorph or Form 3 polymorph, from omadacycline freebase are known in the art and are described, e.g., in U.S. Pat. No. 8,383,610, the entire contents of which are incorporated herein by reference.

In some examples, the method of preparing a tosylate salt of omadacycline from a solution comprising crude omadacycline freebase comprises
subjecting a solution comprising crude omadacycline freebase to purification by HPLC, thereby obtaining a solution comprising HPLC-purified omadacycline freebase;
concentrating the solution comprising HPLC-purified omadacycline freebase using nanofiltration, thereby obtaining a concentrated solution comprising HPLC-purified omadacycline freebase; and
crystallizing omadacycline freebase from the concentrated solution comprising HPLC-purified omadacycline freebase, thereby producing omadacycline crystalline freebase; and
reacting omadacycline crystalline freebase in a tosylation reaction, thereby obtaining a tosylate salt of omadacycline.

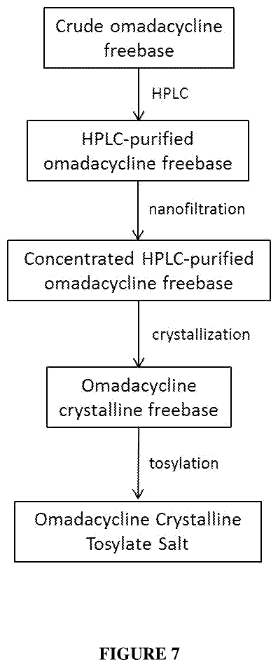

A scheme illustrating the procedure for preparing a tosylate salt of omadacycline as described above is shown in FIG. 7.

The step of subjecting a solution comprising crude omadacycline freebase to purification by HPLC, thereby obtaining a solution comprising HPLC-purified omadacycline freebase may be carried out as described herein in the preceding sections.

The step of concentrating the solution comprising HPLC-purified omadacycline freebase using nanofiltration, thereby obtaining a concentrated solution comprising HPLC-purified omadacycline freebase may be carried out as described herein in the preceding sections.

The step of crystallizing the omadacycline freebase from the concentrated solution comprising HPLC-purified omadacycline freebase, thereby producing omadacycline crystalline freebase may be carried out as described herein in the preceding sections.

The present invention also provides a tosylate salt of omadacycline obtained by the method as described above. In some examples, the tosylate salt of omadacycline is a crystalline tosylate salt of omadacycline, e.g., Form 1 polymorph, Form 2 polymorph or Form 3 polymorph of the crystalline tosylate salt of omadacycline as described in U.S. Pat. No. 8,383,610.

In some embodiments, the present invention also provides a pharmaceutical composition comprising tosylate salt of omadacycline, wherein the tosylate salt of omadacycline is obtained by the method as described above. The tosylate salt of omadacycline may be a crystalline tosylate salt of omadacycine, e.g., Form 1 polymorph, Form 2 polymorph or Form 3 polymorph of crystalline tosylate salt of omadacycline. Exemplary pharmaceutical composition comprising tosylate salt of omadacycline are described, e.g., in U.S. Pat. No. 9,314,475, the entire contents of which are incorporated herein by reference.

The present invention also provides methods for treating or preventing a bacterial infection in a subject in need thereof that comprise administering to the subject tosylate salt of omadacycline that has been prepared according to methods of the present invention. Also provided herein are methods for treating or preventing a bacterial invention in a subject in need thereof that comprise administering to the subject a pharmaceutical composition comprising tosylate salt of omadacycline that has been prepared using methods of the invention.

In some examples, the bacterial infection may be caused by a Gram-positive or a Gram-negative bacteria. In some examples, the bacterial infection may be caused by a bacteria that is resistant to other tetracycline compounds. In some examples, the bacterial infection may be caused by a bacteria of a species selected from the group consisting of K. pneumoniae, Salmonella, E. hirae, A. baumanii, B. catarrhalis, H. influenza, P. aeruginosa, E. faecium, E. coli, S. aureus and E. faecalis.

In one example, the bacterial infection is an acute bacterial skin structure infection (ABSSSI). In some embodiments, ABSSSI may be caused by a Gram-positive or a Gram-negative bacteria, e.g., a bacteria of a species selected from the group consisting of Staphylococcus aureus (methicillin-susceptible and -resistant isolates), including cases with concurrent bacteremia, Staphylococcus lugdunensis, Streptococcus pyogenes, Streptococcus agalactiae, Streptococcus anginosus grp. (includes S. anginosus, S. intermedius, and S. constellatus), Streptococcus mitis, Enterococcus faecalis (vancomycin-susceptible isolates), Enterobacter cloacae, Klebsiella pneumoniae, Prevotella melaninogenica, and Finegoldia magna.

In another example, the bacterial infection may be a community-acquired bacterial pneumonia (CABP). In some embodiments, CABP may be caused by a Gram-positive, a Gram-negative or an atypical bacteria, e.g., a bacteria of a species selected from the group consisting of Streptococcus pneumoniae (penicillin-susceptible and -resistant isolates, macrolide-resistant isolates), including cases with concurrent bacteremia, Staphylococcus aureus (methicillin-susceptible isolates), Haemophilus influenzae (beta-lactamase negative and positive isolates), Haemophilus parainfluenzae, Klebsiella pneumoniae, Legionella pneumophila, Mycoplasma pneumoniae, and Chlamydophila pneumoniae.

In another example, the bacterial infection is a C. difficile infection.

In yet another example, the bacterial infection may be a urinary tract infection (UTI).

### EXEMPLIFICATION OF THE INVENTION

### Example 1. Crystallization of Omadacycline Free Base from Acetone

This example illustrates a typical procedure for crystallizing free base of omadacycline from acetone and characterizing the crystalline product. Free base of omadacycline was crystallized from a retentate obtained after nanofiltration. The retentate with a concentration of 80-100 mg/mL was diluted with the same volume of acetone, seeded and stirred, causing crystallization of omadacycline free base. The product was filtered off and dried.

The crystalline free base of omadacycline was characterized by an XRPD analysis, and the resulting XRPD spectrum is shown in FIG. 1. Significant peaks (° 20) present in the XRPD spectrum in FIG. 1 are listed in the Table 2 below.

TABLE 2
Significant peaks in the XRPD spectrum of
crystalline free base of omadacycline

| Peak (°2θ) | Intensity (I/Iₒ) |
|---|---|
| 7.25 | 100 |
| 7.37 | 78 |
| 10.33 | 28 |
| 12.58 | 62 |
| 12.81 | 39 |
| 14.75 | 38 |
| 16.44 | 63 |
| 17.86 | 88 |
| 19.32 | 60 |
| 19.44 | 48 |
| 19.62 | 27 |
| 22.19 | 36 |
| 23.38 | 26 |
| 24.33 | 35 |

Crystallization improved the overall purity of the omadacycline free base, especially with regard to the removal of the β-epimer, as is evident from the data shown in Table 3.

TABLE 3
Content of β-epimer during purification of omadacycline free base

| Purification Stage | Content of β-epimer (% a/a) |
|---|---|
| Crude omadacycline free base | 11.13 |
| Pooled fractions after preparative HPLC | 1.71 |
| Crystalline omadacycline free base | 0.90 |

### Example 2. Identification of Additional Solvents for Crystallizing Omadacycline Free Base

The goal of this experiment was to identify additional solvents beside acetone that may be used for crystallizing omadacycline free base.

### Material and Methods

The starting material used was the amorphous free base prepared by dissolving purified free base from different batches of omadacycline in dilute hydrochloric acid. The base was extracted by dichloromethane (DCM) at neutral pH and the extracts were concentrated and dried. The resulting material was an orange powder with an area % of purity of 98.22% and a β-epimer level of 1.44% as determined by HPLC.

Initial attempts of crystallization were done on 0.5 gram scale of omadacycline and typical volume of solvent was 10 mL. For crystallization of amorphous omadacycline, 41 different solvents or combinations of solvents were tested. For binary combinations of solvents the content of second solvent in most cases did not exceed 5%. The solution mixtures of omadacycline were stirred overnight at room temperature. Any solid that formed was filtered off, washed and dried. In each case, samples of solid and filtrate were analyzed by HPLC and compared with the starting material. Conclusions about the crystallinity of the product was based on the HPLC results; crystalline product should be more pure then the starting material.

Based on these initial HPLC result, three solvents for crystallization were selected and used for another crystallization at a 1 gram scale. These three solvents which included 5% of water in all cases were isopropanol, acetonitrile and 2-butanone (methyl ethyl ketone). Confirmation of crystallinity was then done by XRPD analysis on the product of these three crystallizations and was compared to a control crystalline free base of omadacycline.

### Results

Table 4 below contains comparison of the HPLC purity between the starting material and the three products. Output yields are also given. All solvents used for crystallization contained 5% of water.

TABLE 4
Yield and purity results

| | Omadacycline | β-epimer | Yield |
|---|---|---|---|
| Material | (%) | (%) | (%) |
| Starting Material | 98.22 | 1.44 | NA |
| Product of crystallization | 99.56 | 0.21 | 92 |
| from isopropanol | | | |
| Product of crystallization | 99.30 | 0.43 | 89 |
| from acetonitrile | | | |
| Product of crystallization | 99.67 | 0.25 | 79 |
| from 2-butanone | | | |

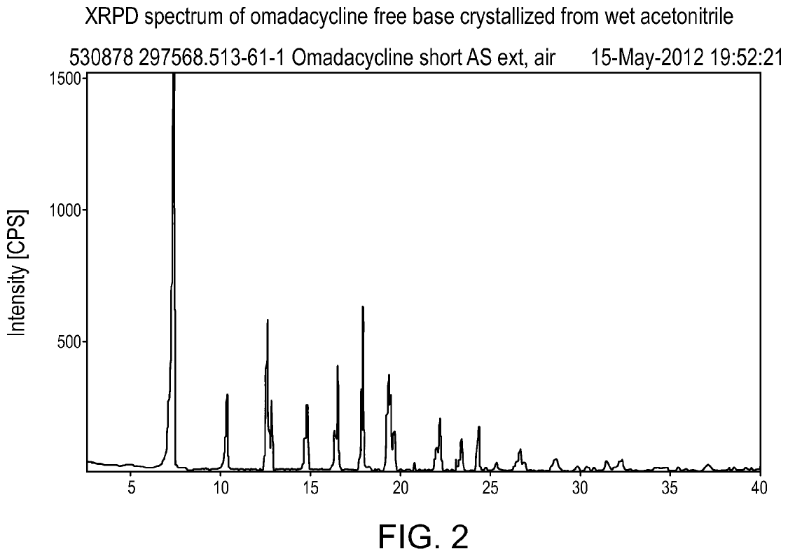

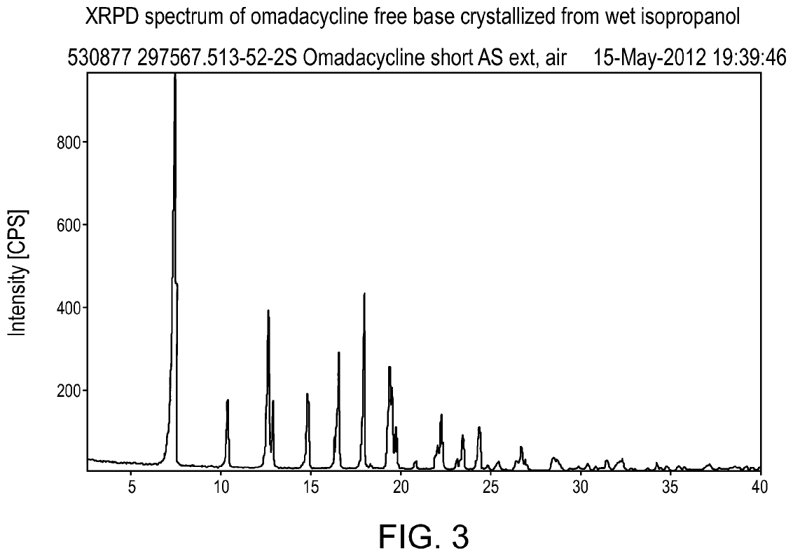

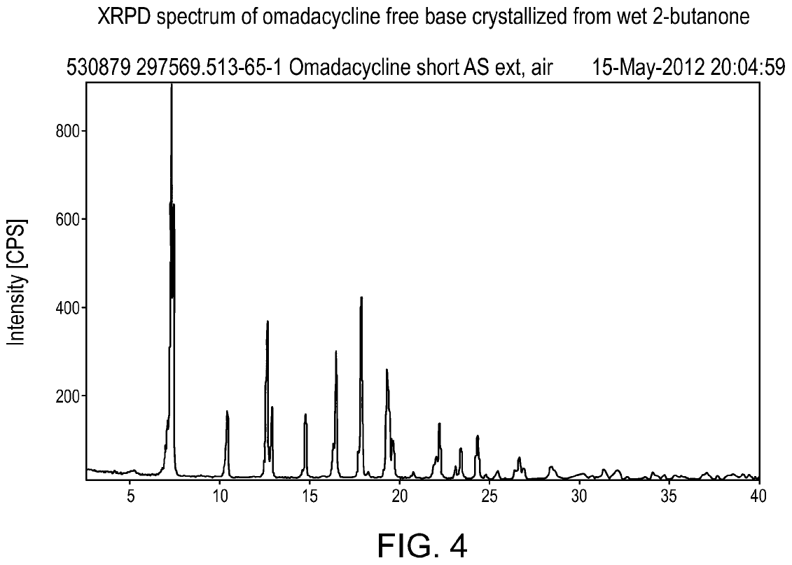

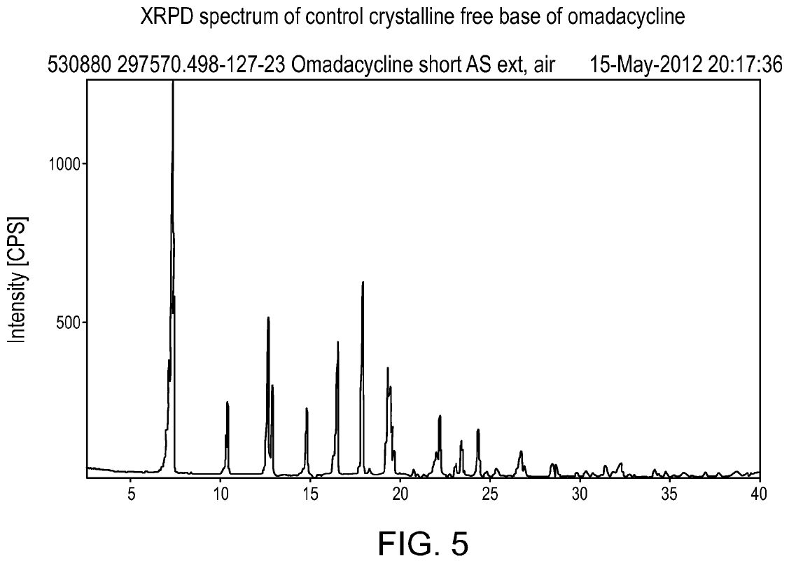

Confirmation of crystallinity was done by XRPD analysis on the products of crystallization listed in Table 4. FIG. 2 shows XRPD spectrum of omadacycline free base crystallized from wet acetonitrile. FIG. 3 shows XRPD spectrum of omadacycline free base crystallized from wet isopropanol. FIG. 4 shows XRPD spectrum of omadacycline free base crystallized from wet 2-butanone. FIG. 5 shows XRPD spectrum of control crystalline free base of omadacycline. As is evident by comparing FIGS. 1-3 to FIG. 4, products of crystallization from wet acetonitrile, wet isopropanol and wet 2-butanone were crystalline.

### Example 3. Use of Nanofiltration for Concentration of Aqueous Solution of Omadacycline

The goal of this experiment was to demonstrate that nanofiltration can be effectively used to concentrate aqueous solution of omadacycline free base after purification by preparative HPLC. In a typical experiment, 150 grams of crude free base of omadacycline (purity of 67.4% w/w, 101.1 gram adjusted to assay) was purified by HPLC. For further processing, a fraction was selected that contained 80.34 g of omadacycline in 4070 ml of 93:7 mixture water/acetonitrile (concentration of 19.74 mg/mL). This solution was concentrated by nanofiltration in two portions, 2000 mL and 2070 mL. As an output, two retentate solutions with concentrations of 80.67 mg/mL and 52.46 mg/mL were obtained containing a total of 76.09 grams of the product. Each retentate solution was subjected to extraction with DCM to afford omadacycline as a dry powder; 71.04 grams corrected for the assay. In total, 892 mL of DCM was used for the extractive recovery. In comparison, the process utilizing extraction with DCM to concentrate aqueous solution of omadacycline requires approximately 43.97 L of DCM for the same amount of product. A further reduction in DCM volume would be possible if the second portion of the nanofiltration were concentrated to the same level as the first (i.e., ˜80 mg/mL).

It is noted that subjecting the retentate solution obtained as a result of nanofiltration to extraction with DCM is optional. As described in Example 4 below, free base of omadacycline may be crystallized directly from the retentate solution to produce crystalline omadacycline freebase.

In the next step, omadacycline free base was subjected to the tosylation/crystallization procedure to give tosylate salt in the amount of 82.85 grams (59.17 grams adjusted for assay). Purity of the product was within the specifications.

### Example 4. Production of Crystalline Tosylate Salt of Omadacycline Via Crystalline Free Base

The goal of this experiment was to produce crystalline tosylate salt of omadacycline via crystalline free base. Crystallization of the free base of omadacycline was carried out using a retentate obtained after purification of the crude free base by preparative HPLC and nanofiltration containing 26.4 kg of crude omadacycline. The retentate was transferred to a reactor, to which the same volume of acetone was added, and the solution was heated to 20-25° C. Using acetic acid/trimethylamine (AcOH/TEA), pH of the solution was adjusted to 7.8-8.0, and seed crystals in acetone/water mixture were added. Reaction mixture was stirred at 20-25° C. for 8-12 hours, and was filtered. Yellow solid was washed with acetone-water mixture and dried. Corrected yield of crystalline free base of omadacycline was 10.62 kg (41.8%).

To prepare crystalline tosylate salt of omadacycline, a solution of p-toluenesulfonic acid in acetone/water was prepared in a glass reactor. In parallel, in a second reactor, a solution containing 9.76 kg of the crystalline free base of omadacycline in dry acetone was prepared at a temperature of 25-30° C. Subsequently, 20% of the volume of the p-toluenesulfonic acid solution was added to the second reactor at a temperature of 25-30° C. The reaction mixture was seeded, and the remainder of the p-toluenesulfonic acid solution was transferred over the period of 2-3 hours. The resulting suspension was stirred for 1-3 hours at 10-15° C., filtered off, washed and dried. Yield of the crystalline tosylate salt of omadacycline was 11.78 kg (92.2%).

### EQUIVALENTS

Those skilled in the art will recognize, or be able to ascertain using no more than routine experimentation, many equivalents to the specific embodiments and methods described herein. Such equivalents are intended to be encompassed by the scope of the present invention. All patents, patent applications, and literature references cited herein are hereby expressly incorporated by reference.

1. A crystalline form of freebase of omadacycline, wherein omadacycline is represented by formula (1):

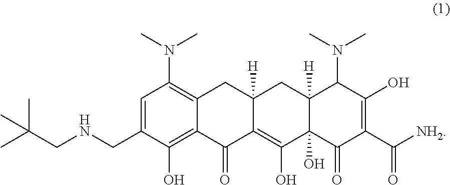 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]

2. The crystalline form of claim 1, wherein the omadacycline is represented by formula (2):

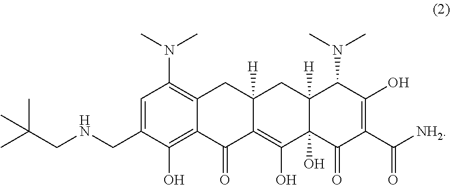 [SMILES: CN(C)c1cc(CNCC(C)(C)C)c(O)c2c1C[C@H]1C[C@H]3[C@H](N(C)C)C(O)=C(C(N)=O)C(=O)[C@@]3(O)C(O)=C1C2=O]

3. A polymorph of the crystalline form of claim 1, characterized by an X-ray powder diffraction pattern that includes at least one peak selected from the group consisting of:
a peak at approximately 7.25° 2θ;
a peak at approximately 7.37° 2θ;
a peak at approximately 10.33° 2θ;
a peak at approximately 12.58° 2θ;
a peak at approximately 12.81° 2θ;
a peak at approximately 14.75° 2θ;
a peak at approximately 16.44° 2θ;
a peak at approximately 17.86° 2θ;
a peak at approximately 19.32° 2θ;
a peak at approximately 19.44° 2θ;
a peak at approximately 19.62° 2θ;
a peak at approximately 22.19° 2θ; and
a peak at approximately 23.38° 2θ.

4-5. (canceled)

6. A method of preparing the polymorph of claim 3, comprising crystallizing freebase form of omadacycline from a solvent system that comprises an organic solvent and water.

7. The method of claim 6, wherein the organic solvent and water are present in the solvent system at a ratio ranging from about 5:95 v/v to about 95:5 v/v organic solvent:water.

8. The method of claim 7, wherein the organic solvent is selected from the group consisting of acetonitrile, acetone, isopropyl alcohol and methyl ethyl ketone.

9-20. (canceled)

21. A method of purifying freebase form of omadacycline, wherein the omadacycline is represented by formula (1): 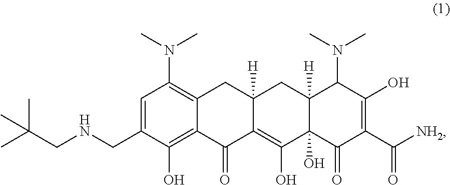 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]
said method comprising
subjecting a solution comprising crude freebase form of omadacycline to purification by high performance liquid chromatography (HPLC)
wherein the HPLC comprises the use of a modifier selected from the group consisting of a weak acid and an organic amine, thereby obtaining a solution comprising HPLC-purified freebase form of omadacycline.

22-40. (canceled)

41. A method of preparing a tosylate salt of omadacycline, wherein the omadacycline is represented by formula (1): 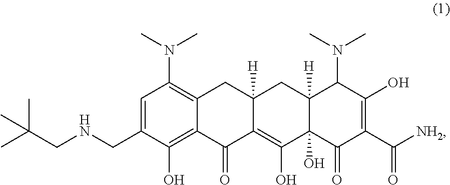 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]
said method comprising:
purifying a freebase form of omadacycline by the method of claim 21, thereby obtaining a purified freebase form of omadacycline; and
reacting said purified freebase form of omadacycline in a tosylation reaction, thereby obtaining a tosylate salt of omadacycline.

42. A method of preparing a tosylate salt of omadacycline, wherein the omadacycline is represented by formula (1): 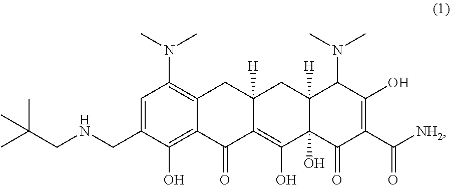 [SMILES: CN(C)c1cc(CNCC(C)(C)C)c(O)c2c1C[C@H]1C[C@H]3C(N(C)C)C(O)=C(C(N)=O)C(=O)[C@@]3(O)C(O)=C1C2=O]
said method comprising:
crystallizing freebase form of omadacycline, thereby obtaining a crystalline form of the freebase of omadacycline; and
reacting said crystalline form of the freebase of omadacycline in a tosylation reaction, thereby obtaining a tosylate salt of omadacycline.

43-64. (canceled)

65. A pharmaceutical composition comprising the crystalline form of freebase of omadacycline of claim 1 and a pharmaceutically acceptable carrier.

66-69. (canceled)

70. The pharmaceutical composition of claim 65, wherein the pharmaceutical composition is in a tablet form or wherein the pharmaceutical composition is an injectable formulation in the form of a lyophilized powder.

71. (canceled)

72. A method of treating or preventing a bacterial infection in a subject in need thereof, said method comprising administering to said subject an effective amount of the crystalline form of freebase of omadacycline of claim 1.

73-76. (canceled)

77. The method of claim 72, wherein the bacterial infection is caused by a Gram-positive or a Gram-negative bacteria.

78. The method of claim 72, wherein the bacterial infection is caused by a bacteria that is resistant to other tetracycline compounds.

79. The method of claim 72, wherein the bacterial infection is caused by a bacteria of a species selected from the group consisting of K. pneumoniae, Salmonella, E. hirae, A. baumanii, B. catarrhalis, H. influenza, P. aeruginosa, E. faecium, E. coli, S. aureus and E. faecalis.

80. The method of claim 72, wherein the bacterial infection is an acute bacterial skin structure infection (ABSSSI) or a community-acquired bacterial pneumonia (CABP).

81. The method of claim 80, wherein the ABSSSI is caused by a bacteria of a species selected from the group consisting of Staphylococcus aureus (methicillin-susceptible and -resistant isolates), including cases with concurrent bacteremia, Staphylococcus lugdunensis, Streptococcus pyogenes, Streptococcus agalactiae, Streptococcus anginosus grp. (includes S. anginosus, S. intermedius, and S. constellatus), Streptococcus mitis, Enterococcus faecalis (vancomycin-susceptible isolates), Enterobacter cloacae, Klebsiella pneumoniae, Prevotella melaninogenica, and Finegoldia magna.

82. (canceled)

83. The method of claim 80, wherein the CABP is caused by a bacteria of a species selected from the group consisting of Streptococcus pneumoniae (penicillin-susceptible and -resistant isolates, macrolide-resistant isolates), including cases with concurrent bacteremia, Staphylococcus aureus (methicillin-susceptible isolates), Haemophilus influenzae (beta-lactamase negative and positive isolates), Haemophilus parainfluenzae, Klebsiella pneumoniae, Legionella pneumophila, Mycoplasma pneumoniae, and Chlamydophila pneumoniae.

84. The method of claim 72, wherein the bacterial infection is caused by a bacterial of a species C. difficile or a mycobacteria.

85. (canceled)
